(19) United States
(12) Patent Application Publication
(10) Pub. No.: US 2023/0084585 A1
(43) Pub. Date: Mar. 16, 2023

(54) ELECTRODE ASSEMBLY, SYSTEMS, AND METHODS OF USE THEREOF

(71) Applicant: Stryker Corporation, Kalamazoo, MI (US)

(72) Inventors: Dennis Changmin Sohn, Kirkland, WA (US); Tyson G. Taylor, Bothell, WA (US); Doan Huu Dinh, Seattle, WA (US); Daniel W Piraino, Seattle, WA (US); Shardul Varma, Kirkland, WA (US); Karl Frederick Gauglitz, Snohomish, WA (US); Robert G. Walker, Seattle, WA (US)

(21) Appl. No.: 17/944,628

(22) Filed: Sep. 14, 2022

Related U.S. Application Data
(60) Provisional application No. 63/244,089, filed on Sep. 14, 2021.

(51) Int. Cl. A61N 1/04

(52) U.S. Cl. CPC A61N 1/046; A61N 1/0492; A61B 5/053

(57) ABSTRACT

An electrode assembly that is adjustable in size, as well as systems, and methods of use thereof are described. The electrode assembly includes multiple electrode portions including at least a first electrode portion and a second electrode portion, the second electrode portion disposed on the first electrode portion at an edge of the first electrode portion, wherein the second electrode portion has a cutout, and wherein the first electrode portion spans the cutout. The electrode assembly is configured to be used with a medical device, such as an external defibrillator. A process of automatically selecting a usage mode of a medical device based on detecting which electrode portion(s) has been removed from an electrode storage tray is also described.

### CROSS-REFERENCE TO RELATED APPLICATION

This application claims the benefit of U.S. Provisional Application No. 63/244,089, titled “An Electrode Assembly, Systems, and Methods of Use Thereof” and filed on Sep. 14, 2021, and which is incorporated by reference herein in its entirety.

### BACKGROUND

A defibrillator is a medical device configured to administer defibrillation therapy to a patient through electrodes. Because patients vary in size, users of defibrillators have to maintain multiple sets of different-sized electrodes. For example, a set of pediatric-sized electrodes have to be maintained in addition to a separate set of adult-sized electrodes in case a child is in need of defibrillation therapy. Due to the limited shelf life of pediatric-sized electrodes and due to the rarity of their use, pediatric-sized electrodes are often thrown away before they are ever used. The alternative of using a single set of electrodes having a fixed size to provide defibrillation therapy to a variety of different-sized patients is ill-advised. This is because such a single set of electrodes, while suitable for some patients, are not optimized for a variety of different-sized patients. For example, using electrodes that are too big for a pediatric patient is not as effective as using electrodes that are optimally-sized for children. Likewise, using electrodes that are too small for an adult patient is not as effective as using electrodes that are optimally-sized for adults. The disclosure made herein is presented with respect to these and other considerations.

### BRIEF DESCRIPTION OF THE DRAWINGS

FIG. 1 illustrates a perspective exploded view of an example electrode assembly having two electrode portions.

FIG. 2 illustrates a side view of the example electrode assembly depicted in FIG. 1.

FIG. 3 illustrates a top view of the example electrode assembly depicted in FIG. 1.

FIG. 4 illustrates a top view of the example electrode assembly depicted in FIG. 1, and further depicting a user removing a first electrode portion of the electrode assembly from a second electrode portion of the electrode assembly.

FIG. 5 illustrates a bottom view of two electrode portions of the electrode assembly depicted in FIG. 1, the two electrode portions detached from one another and positioned in a side-by-side arrangement in FIG. 5.

FIG. 6 illustrates a bottom view of the electrode assembly depicted in FIG. 1, and further depicting a perforated layer of gel disposed on the respective conductive areas of the electrode portions.

FIG. 7 illustrates a perspective exploded view of another example electrode assembly having two electrode portions, the example electrode assembly including conductive elements and a conductive film.

FIG. 8 illustrates a perspective exploded view of another example electrode assembly having two electrode portions, the example electrode assembly including an adhesive backing.

FIG. 9 illustrates another example electrode assembly having four electrode portions, each electrode portion being removable to accommodate a variety of different-sized patients.

FIG. 10 illustrates an electrical cable of an electrode assembly having a measurement scale printed thereon, and a technique for determining which electrode portion(s) to use on a patient based on a portion of the patient's body measured with the measurement scale.

FIG. 11 illustrates a set of electrode assemblies disposed in an electrode storage tray of an external defibrillator, and further depicting a user removing a first electrode portion of an electrode assembly from the electrode storage tray.

FIG. 12 illustrates the example external defibrillator depicted in FIG. 11 outputting a notification that the external defibrillator is in a pediatric mode.

FIG. 13 illustrates a close-up view of the display of the example external defibrillator depicted in FIG. 11, the display outputting an indication that a user-selected mode is incompatible with the removed electrode portion, and a request for the user to acknowledge the incompatibility.

FIG. 14 illustrates a close-up view of the display of the example external defibrillator depicted in FIG. 11, the display outputting an indication that a user-selected mode is incompatible with the removed electrode portion, and a prompt for the user to re-attach an additional electrode portion.

FIG. 15 illustrates a side cross-sectional view of an example external defibrillator, and further depicting an optical sensor disposed in the electrode storage tray, the optical sensor being usable for determining whether an electrode portion of the electrode assembly remains disposed in the electrode storage tray.

FIG. 16 illustrates a perspective view of an example external defibrillator, and further depicting sense leads of an electrical circuit disposed in the electrode storage tray, the electrical circuit being usable for determining whether an electrode portion of the electrode assembly remains disposed in the electrode storage tray.

FIG. 17 illustrates a side cross-sectional view of an example external defibrillator, and further depicting a mechanical switch disposed in the electrode storage tray, the mechanical switch being usable for determining whether an electrode portion of the electrode assembly remains disposed in the electrode storage tray.

FIG. 18 illustrates an example process for adapting an electrode assembly to a size that is suitable for delivering therapy to a patient.

FIG. 19 illustrates an example process for selecting a usage mode of a medical device based on the removal of one or more electrode portions of an electrode assembly from an electrode storage tray of the medical device.

FIG. 20 illustrates an example external defibrillator having an electrode assembly described herein and configured to perform various functions described herein.

### DETAILED DESCRIPTION

The disclosure provides an electrode assembly that includes multiple electrode portions. The multiple electrode portions include at least a first electrode portion and a second electrode portion. The second electrode portion is disposed on the first electrode portion at an edge of the first electrode portion. The second electrode portion has a cutout, and the first electrode portion spans the cutout. The electrode assembly is configured to be used with a medical device, such as an external defibrillator. In an example, the electrode assembly is coupled to a medical device, and a user of the medical device uses the electrode assembly to deliver therapy to a patient. The entire electrode assembly is usable to deliver therapy to a patient who is greater than a threshold size and/or age. In order to deliver therapy to a patient who is less than a threshold size and/or age, the user is able to remove the first electrode portion from the second electrode portion and use the first electrode portion (without the second electrode portion) to deliver therapy to the smaller and/or younger patient. In this manner, the disclosed electrode assembly is convertible (e.g., by removing one or more electrode portions from the electrode assembly) into an electrode of a size that is suitable for the size and/or age of the patient who is to receive electrode therapy via the medical device. The number of discrete sizes to which a user is able to adjust the electrode assembly corresponds to the number of electrode portions included in the electrode assembly.

This electrode assembly (sometimes referred to herein as a “universal” electrode or “all-in-one” electrode) reduces the variety and cost of inventory for users by eliminating the need to maintain a separate set of electrodes for patients who are in separate size and/or age ranges. For example, a user of a medical device need not maintain a separate set of pediatric-sized electrodes for use with a medical device, which are often thrown away before they are ever used. Instead, if the entire electrode assembly is too large for a particular patient (e.g., an infant patient, a pediatric patient, etc.), the user is able to remove one or more electrode portions from the disclosed electrode assembly to create an electrode of a suitable size for delivering therapy to the patient. This way, the electrode assembly is usable as-is for larger-sized patients, and the electrode assembly is convertible to an appropriately-sized electrode for delivering therapy to smaller-sized patients. In addition to its use across a wide variety of patient types, the disclosed electrode assembly is also usable across a wide variety of medical device types and/or models, such as different types and/or models of external defibrillators.

This disclosure also provides systems and devices including the electrode assembly disclosed herein, as well as processes for changing the size of the electrode assembly by removal of one or more electrode portions therefrom. Furthermore, this disclosure provides processes implemented by a medical device, such as an external defibrillator, for delivering therapy to a patient using the disclosed electrode assembly, as well as processes for selecting a usage mode of a plurality of usage modes in which the medical device is to operate based on the medical device detecting which electrode portion(s) has been removed from the electrode assembly. The electrode assembly disclosed herein, when used with a medical device, improves the performance of delivering therapy to a patient in part because the electrode assembly is adaptable to a more appropriate size for the patient. In the example of an electrode assembly configured to be used with an external defibrillator, this allows for delivering a more appropriate amount of energy to deliver an electrical shock to the patient through an appropriately-sized conductive area(s) of the electrode portion(s).

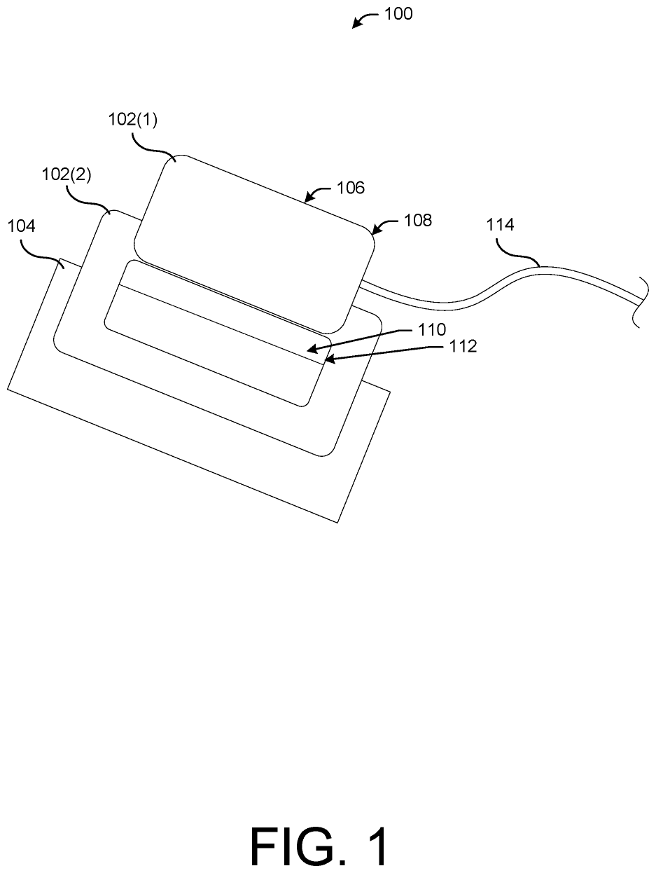

FIG. 1 illustrates a perspective exploded view of an example electrode assembly 100 having two electrode portions 102. The two electrode portions 102 include a first electrode portion 102(1) and a second electrode portion 102(2). In the example of FIG. 1, the electrode assembly 100 further includes a liner 104 (e.g., a plastic liner) that is configured to be disposed on a layer of gel, the layer of gel being disposed on one or more conductive areas on the bottom of the electrode assembly 100 depicted in FIG. 1, as will be described in more detail below. The liner 104 is configured to protect the layer of gel from drying out prior to use of the electrode assembly 100. In other examples, a liner 104 is omitted from the electrode assembly 100. For example, the electrode portions 102 of the electrode assembly 100 may be disposed in an electrode storage tray without a liner to store the electrode assembly 100 and before using it to deliver therapy to a patient.

The electrode assembly 100 is configured to be implemented with (e.g., as part of, as an accessory of, etc.) any suitable type of device, such as a medical device. In some examples, the electrode assembly 100 is to be implemented with an emergency medical device, such as a defibrillator (e.g., an external defibrillator) and/or a patient monitor. It is to be appreciated that, in other examples, the electrode assembly 100 is usable with a non-medical device, such as an electrical muscle stimulation (EMS) suit. Nevertheless, the examples provided herein pertain to using the electrode assembly 100 with a medical device, and, specifically, an external defibrillator. These examples are to illustrate the various features of the electrode assembly 100 without limiting the use of the electrode assembly 100 to any particular application.

According to some examples, the electrode assembly 100 represents one assembly of two electrode assemblies that constitute a set (e.g., a pair) of electrode assemblies that are usable with a medical device to deliver therapy to a patient. For example, the electrode assembly 100, or a portion 102 thereof, is configured to be attached to a first portion of a patient's body, such as the right infraclavicular region of the patient to one side of the heart. Meanwhile, the other electrode assembly of the set (not shown in FIG. 1), or a portion 102 thereof, is configured to be placed on a second portion of the patient's body, such as the left inferolateral chest region of the patient to the other side of the heart. With the set of electrode assemblies attached to the patient, the medical device provides therapy to the patient via the attached electrode assemblies. The electrode assembly 100 is sometimes shortened to “electrode 100” herein, although it is to be appreciated that, when referred to herein as an “electrode 100,” the electrode 100 can be made up of multiple electrode portions, such as the first electrode portion 102(1) and the second electrode portion 102(2) (collectively 102) depicted in FIG. 1. It is also to be appreciated that the electrode assembly 100 is convertible into a single electrode portion 102. Furthermore, the electrode assembly 100 and/or an individual electrode portion 102 thereof is sometimes referred to herein as an “electrode pad” or an “adhesive electrode.”

In some examples, the individual electrode portions 102 of the electrode assembly 100 are made, at least in part, of a flexible material, such as a foam, silicone, or plastic material. In this way, the individual electrode portions 102 are configured to conform to the body of the patient when they are attached to the patient for improved contact and/or adhesion between the skin and the layer of gel that is disposed on the conductive areas of the electrode portion(s) 102.

FIG. 1 depicts the electrode portions 102 as having a substantially rectangular shape, but this is merely an example shape. It is to be appreciated that the individual electrode portions 102 may have any suitable geometric shape including a circular shape, a triangular shape, a quadrilateral (e.g., square, rectangle, trapezoid, parallelogram, rhombus, rhomboid, etc.) shape, a pentagonal shape, a hexagonal shape, a heptagonal shape, an octagonal shape, and/or any suitable polygonal shape. Moreover, in some examples the shapes of the respective electrode portions 102 of the electrode assembly 100 differ. In other examples, such as the example electrode assembly 100 depicted in FIG. 1, the shapes of the respective electrode portions 102 may be substantially similar. Accordingly, an individual electrode portion 102 has one or more edges 106, where the number of edges 106 depends on the shape of the electrode portion 102. For example, the first electrode portion 102(1) depicted in FIG. 1 has four outer edges 106 because it is rectangular in shape. Similarly, the second electrode portion 102(2) depicted in FIG. 1 has four outer edges 106. These outer edges 106 are at a periphery of the respective electrode portion 102. The individual electrode portions 102 depicted in FIG. 1 also have four corners 108 because they are each rectangular in shape. In some examples, the corners 108 of the electrode portions 102 are rounded or curved, which helps to prevent burns to the patient. This is because a sharp corner causes electrical current to concentrate at a location on the conductive area of the electrode portion 102, which causes skin that is in contact with that location to burn. One advantage of electrode portions 102 having a circular shape is that burns to the patient are minimized due to the absence of corners. One advantage of electrode portions 102 having a rectangular shape is that they minimize the material used for product packaging. Moreover, an electrode storage tray of a medical device is able to store multiple rectangular electrode assemblies 100 in a side-by-side arrangement to reduce (e.g., minimize) the material of the electrode storage tray.

The overall size of each electrode portion 102 of the electrode assembly 100 is configurable to accommodate patients that span a desired size and/or age range. In an example, the first electrode portion 102(1) is of a size that is appropriate for use by itself (e.g., without the second electrode portion 102(2)) to deliver therapy to a patient who is less than a threshold size and/or age, such as a pediatric patient. FIG. 1 depicts the first electrode portion 102(1) as having smaller length and width dimensions than the corresponding length and width dimensions of the second electrode portion 102(2). In other words, the overall area (without considering cutouts, holes, etc.) of the first electrode portion 102(1) is smaller than the second electrode portion 102(2). In some examples, the first electrode portion 102(1) has a width (e.g., a shorter side edge 106) of about 7.5 to 10 centimeters (cm) (3 to 4 inches (in)), and a length (e.g., a larger side edge 106) of about 12.5 to 15 cm (5 to 6 in). In an example where the electrode portions 102 are circular in shape, the outer diameter of the first electrode portion 102(1) is smaller than the outer diameter of the second electrode portion 102(1).

FIG. 1 further depicts the second electrode portion 102(2) as having a cutout 110. In the example of FIG. 1, the cutout 110 defined in the second electrode portion 102(2) has a rectangular shape, but this is merely an example shape. In some examples, the area of the cutout 110 is smaller than the area of the first electrode portion 102(1). In this manner, the first electrode portion 102(1) spans the cutout 110 of the second electrode portion 102(2) and overlaps a portion of the second electrode portion 102(2). The difference in size between the cutout 110 and the first electrode portion 102(1) is relatively small, in some examples, such that the first electrode portion 102(1) overlaps the second electrode portion 102(2) by a relatively small amount of overlap. This overlap 300 is depicted in FIG. 3. In some examples, the amount of overlap 300 is about 0.5 to 2.5 cm (0.25 to 1 in), but this is merely an example. FIG. 3 depicts an example where the first electrode portion 102(1) completely spans the cutout 110. In some examples, there are one or more gaps between the first electrode portion 102(1) and the second electrode portion 102(2) where the cutout 110 is not covered by the first electrode portion 102(1). In these examples, the first electrode portion 102(1) may substantially span the cutout 110 even though there may be a gap(s) where the cutout 110 is left uncovered by the first electrode portion 102(1). In some examples, the first electrode portion 102(1) substantially spans the cutout 110 if more than a threshold amount of the cutout 110 is covered by the first electrode portion 102(1), such as more than 50% of the cutout 110, more than 75% of the cutout 110, or more than 90% of the cutout 110 is covered by the first electrode portion 102(1). As used herein, “span” the cutout 110 can mean completely span or substantially span.

Referring again to FIG. 1, an inner edge(s) 112 of the second electrode portion 102(2) defines the cutout 110 (sometimes referred to herein as a “window 110”). The rectangular cutout 110 depicted in FIG. 1 creates a second electrode portion 102(2) in the form of a rectangular frame. For a second electrode portion 102(2) that has a circular outer shape and a cutout 110 that is also circular, the second electrode portion 102(2) is annular-shaped, or ring-shaped. Furthermore, FIG. 1 depicts the position of the cutout 110 at a center of the second electrode portion 102(2). In other words, the cutout 110 is centered within the second electrode portion 102(2). In this example where the second electrode portion 102(2) has a central cutout 110 defined therein, the electrode portions 102 of the electrode assembly 100 are substantially concentric when the electrode assembly 100 is assembled. In other examples, the centers of the respective electrode portions 102 do not coincide with each other such that the electrode portions 102 are not concentric; instead, the centers of the electrode portions 102 are offset from one another.

FIG. 1 further depicts that the electrode assembly 100 includes an electrical cable 114 (sometimes referred to herein as a “wire 114,” “wire cable 114,” or “lead wire 114”). In the example of FIG. 1, the electrical cable 114 is coupled to the first electrode portion 102(1) at a first end of the electrical cable 114, and is configured to be coupled to a medical device (e.g., an external defibrillator) at a second end of the electrical cable 114. In some examples, one or more electrical signals are sent by the medical device (not shown in FIG. 1) to the first electrode portion 102(1) and/or one or more electrical signals are sent by the first electrode portion 102(2) to the medical device via the electrical cable 114. For example, therapy is deliverable to a patient in the form of an electrical shock delivered from the medical device to the first electrode portion 102(1), and ultimately to the patient via at least the conductive area of the first electrode portion 102(1). The electrical shock is delivered based on an electrical signal(s) received by the first electrode portion 102(1) from an external defibrillator coupled to the other end of the electrical cable 114.

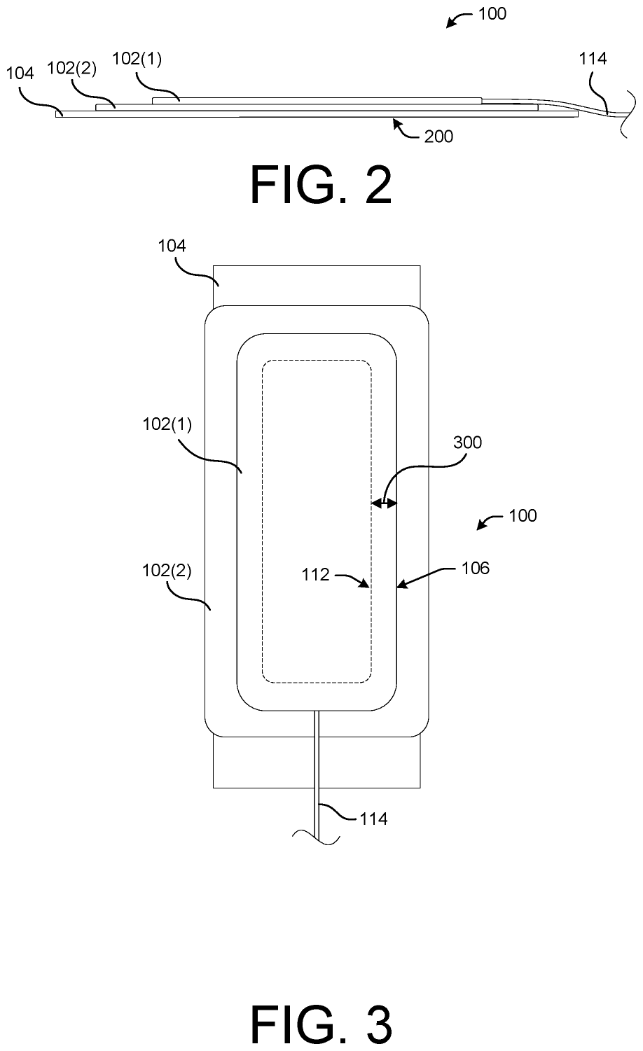

FIG. 2 illustrates a side view of the example electrode assembly 100 depicted in FIG. 1. The electrode assembly 100 is depicted in its assembled configuration in FIG. 2. In some examples, this assembled configuration is the configuration of the electrode assembly 100 prior to its use with respect to a patient. In this configuration, portions of the electrode assembly 100 are stacked atop one another. For example, the second electrode portion 102(2) is stacked atop the liner 104, and the first electrode portion 102(1) is stacked atop the second electrode portion 102(2). Said another way, the liner 104 is disposed on the second electrode portion 102(2) (e.g., a bottom of the second electrode portion 102(2)), and the second electrode portion 102(2) is disposed on the liner 104 (e.g., a top of the liner 104). The second electrode portion 102(2) is also disposed on the first electrode portion 102(1) (e.g., a bottom of the first electrode portion 102(1)). In some examples, the second electrode portion 102(2) is disposed on the first electrode portion 102(1) at a periphery of the first electrode portion 102(1), as illustrated in FIG. 3. In some examples, a top of the second electrode portion 102(2) is disposed on a bottom of the first electrode portion 102(1). Accordingly, the first electrode portion 102(1) is disposed on the second electrode portion 102(2) (e.g., a top of the second electrode portion 102(2)). In yet another way of describing this assembled configuration, the second electrode portion 102(2) is disposed below (or underneath) the first electrode portion 102(1), and the first electrode portion 102(1) is disposed above (or over) the second electrode portion 102(2). The first electrode portion 102(1) is also in contact with the second electrode portion 102(2), and, in the example of FIG. 2, the second electrode portion 102(2) is in contact with the liner 104. In the example of FIG. 2, the second electrode portion 102(2) is also disposed between the liner 104 and the first electrode portion 102(1).

In some examples, the second electrode portion 102(2) is coupled to the first electrode portion 102(1), such as with an adhesive. In the example of an adhesive coupling, the adhesive that couples the electrode portions 102(1) and 102(2) together is interposed between the electrode portions 102(1) and 102(2) where the electrode portions 102 are in contact with each other. In an example, an adhesive (e.g., a non-gelled adhesive) is disposed on a bottom of the first electrode portion 102(1) at a periphery of the first electrode portion 102(1), which allows for an adhesion-based coupling between the electrode portions 102(1) and 102(2). Additionally, or alternatively, the adhesive is disposed on a top of the second electrode portion 102(2) along the inner edge(s) 112 of the second electrode portion 102(2). In some examples, a liner (e.g., a plastic liner) is coupled to the top of the second electrode portion 102(2) along the inner edge(s) 112 of the second electrode portion 102(2) where the first electrode portion 102(1) overlaps the second electrode portion 102(2). Such a liner may be used with the aforementioned adhesive to facilitate removal of the first electrode portion 102(1) from the second electrode portion 102(2). As used herein, the term “couple” may refer to an indirect coupling or a direct coupling between elements. The term “couple,” as used herein, may also refer to a removable coupling or a permanent coupling between the elements. Elements are removably coupled if a user or another entity is able to decouple the elements. Elements are permanently coupled if a user or another entity is unable to decouple the elements without destroying or significantly damaging the elements, or without undue effort to dissemble the elements using tools or machinery. As used herein, the term “couple” can be interpreted as connect, attach, join, engage, interface, link, fasten, or bind. Unless otherwise specified herein, the term “couple” is to be interpreted as coupling elements in a mechanical sense, rather than in an electrical sense, for example. Nevertheless, it is to be appreciated that a mechanical coupling of elements may result in an electrical coupling(s) between multiple elements of the system.

FIG. 3 illustrates a top view of the example electrode assembly 100 depicted in FIG. 1. The electrode assembly 100 is depicted in its assembled configuration in FIG. 3. FIG. 3 also illustrates that the first electrode portion 102(1) spans the cutout 110 of the second electrode portion 102(2) (e.g., the inner edge(s) 112 of the second electrode portion 102(2) is shown in dashed lines in FIG. 3 due to the occlusion of the cutout 110 in FIG. 3). Furthermore, due to the size of the first electrode portion 102(1) being larger than the size of the cutout 110 of the second electrode portion 102(2), the first electrode portion 102(1) overlaps part of the second electrode portion 102(2) by an amount of overlap 300. That is, the outer edge(s) 106 of the first electrode portion 102(1) is not vertically aligned with the inner edge(s) 112 of the second electrode portion 102(2). Rather, the outer edge(s) 106 of the first electrode portion 102(1) is offset from the inner edge(s) 112 of the second electrode portion 102(2) in a lateral dimension. For example, an outer, side edge 106 of the first electrode portion 102(1) is horizontally offset from an inner, side edge 112 of the second electrode portion 102(2) by an amount of overlap 300 depicted in FIG. 3. Furthermore, the outer edge(s) 106 of the first electrode portion 102(1) is horizontally offset from the outer edge(s) 106 of the second electrode portion 102(2). In particular, the outer edge(s) 106 of the first electrode portion 102(1) is inset from the outer edge(s) 106 of the second electrode portion 102(2) such that the second electrode portion 102(2) extends beyond the outer edge(s) 106 of the first electrode portion 102(1).

FIG. 3 also illustrates an example where the first and second electrode portions 102(1) and 102(2) are concentric, or otherwise that the centers of the respective electrode portions 102(1) and 102(2) are coincident. Accordingly, in some examples, the cutout 110 defined in the second electrode portion 102(2) is a central cutout 110 by virtue of its central location with respect to the second electrode portion 102(2). It is to be appreciated however, that the electrode portions 102 are not concentric and/or the centers of the respective electrode portions 102(1) and 102(2) are not coincident in some examples. Nevertheless, in the non-concentric arrangement, the outer perimeter of the smaller electrode portion(s) 102(1) may be surrounded by the outer perimeter of the larger electrode portion(s) 102(2) such that an outer edge(s) 106 of a smaller electrode portion 102(1) is not aligned with and/or does not overhang, or extend beyond, an outer edge(s) 106 of a larger electrode portion 102(2) that is disposed underneath the smaller electrode portion 102(1). In other non-concentric arrangements, one or more outer edges 106 of the smaller electrode portion(s) 102(1) are aligned with and/or overhang, or extend beyond, an outer edge(s) 106 of a larger electrode portion 102(2) that is disposed underneath the smaller electrode portion 102(1). For example, an outer edge 106 of the first electrode portion 102(1) may be aligned with a corresponding outer edge 106 of the second electrode portion 102(2) (e.g., respective outer edges 106 of the electrode portions 102 are flush with each other). In this example configuration where the respective outer edges 106 of the electrode portions 102 are aligned or flush, the cutout 110 may not be enclosed by the material of the material of the larger electrode portion 102(2); instead, the larger electrode portion 102(2) may be a U-shaped electrode portion 102, and after the smaller electrode portion 102(1) is removed from the larger electrode portion 102(2), a layer of gel may bridge the open end of the U-shaped, larger electrode portion 102, or the cutout 110, devoid of gel material, may be exposed.

Notably, the electrode portions 102 of the electrode assembly 100 are not in a side-by-side arrangement when the electrode assembly 100 is in its assembled configuration. Rather, the electrode portions 102 are disposed in a stacked arrangement. That said, in some examples, electrode portions 102 are disposed side-by-side in a coplanar arrangement, and one or more electrode portions 102 are removable from another electrode portion(s) 102, such as by tearing the electrode portions 102 apart along a perforation. Furthermore, in the various examples provided herein, a smaller electrode portion (e.g., the first electrode portion 102(1)) is stacked atop a larger electrode portion (e.g., the second electrode portion 102(2)), and the smaller electrode portion is disposed or otherwise positioned within the outer perimeter of the larger electrode portion. For example, the first electrode portion 102(1) is positioned inside of, or within, the outer perimeter of the second electrode portion 102(2), which spans a larger area than the first electrode portion 102(1), not considering the cutout 110 defined in the second electrode portion 102(2).

Moreover, due to the flexible material (e.g., flexible foam material) of which the substrates of the electrode portions 102 are made, a user is able to press the multiple electrode portions 102 toward the patient until their bottom surfaces come into contact with the patient's skin when the user is placing the electrode assembly 100, or a portion(s) 102 thereof, on the patient. That is, despite the first electrode portion 102(1) being disposed above the second electrode portion 102(2) (See FIG. 2), the thin profile of each electrode portion 102, and the flexibility of the material of which the electrode portions 102 are made allow the user to easily bend and/or flex the electrode portions 102 with minimal pressure applied to the electrode portion(s) 102. This applied pressure causes the bottom surface of the first electrode portion 102(1)—which is exposed through the cutout 110 of the second electrode portion 102(2) when the electrode assembly 100 is in its assembled configuration—is able to be pressed down and into contact with a patient's skin. Accordingly, the layer of gel that is disposed on at least a first conductive area of the first electrode portion 102(1) causes the exposed portion of the first electrode portion 102(1) to remain in contact with the patient's skin due to the adhesive properties of the gel layer. Accordingly, while the stacked electrode portions 102 of the electrode assembly 100 may not be coplanar when the electrode assembly 100 is in its assembled configuration and stored prior to its use, a user can place the electrode assembly 100 on a patient and apply pressure to the electrode assembly 100 to bring the bottom of the first electrode portion 102(1) into contact with the patient's skin, and this causes the electrode portions 102 to become substantially coplanar during delivery of therapy (e.g., defibrillation therapy) to the patient via the electrode assembly 100. The stacked arrangement of the electrode portions 102 and the cutout(s) 110 defined in the larger electrode portion(s) 102 reduces the amount of material used to manufacture the electrode assembly 100, as compared to a design with multiple electrode portions that are arranged side-by-side in the same plane, and/or as compared to a design with multiple stacked electrode portions that are not configured to be used together and are exclusively designed to be used individually/independently to delivery therapy to the patient. For example, the material that would have been used to occupy the cutout 110 of the second electrode portion 102(2) is conserved in the disclosed design of the electrode assembly 100, which reduces the cost of manufacturing and/or conserve resources (e.g., materials).

In order to use the electrode assembly 100, such as to deliver therapy (e.g., defibrillation therapy) to a patient, a user removes (e.g., peels off) the liner 104 to expose a layer of gel between the liner 104 and the remainder of the electrode assembly 100. When the multiple electrode portions 102 are to be used together to deliver the therapy to the patient, the user places the electrode assembly 100 on the patient with the layer of gel facing, and in contact with, the patient. In other words, after removing the liner 104 from the electrode assembly 100 to expose the layer of gel between the liner 104 and electrode portion(s) 102, a bottom 200 (see FIG. 2) of the electrode assembly 100 is placed on the patient. In some examples, a bottom of the second electrode portion 102(2) at a periphery of the second electrode portion 102(2) includes an adhesive (e.g., glue, tape, etc.) to facilitate attachment of the electrode assembly 100 to the patient and/or to improve the contact area between the skin and the layer of gel. The electrode assembly 100 is configured to be used in this manner (e.g., the multiple electrode portions 102 are configured to be used together) to deliver therapy (e.g., defibrillation therapy) to a patient who is greater than a threshold size and/or age, such as an adult patient.

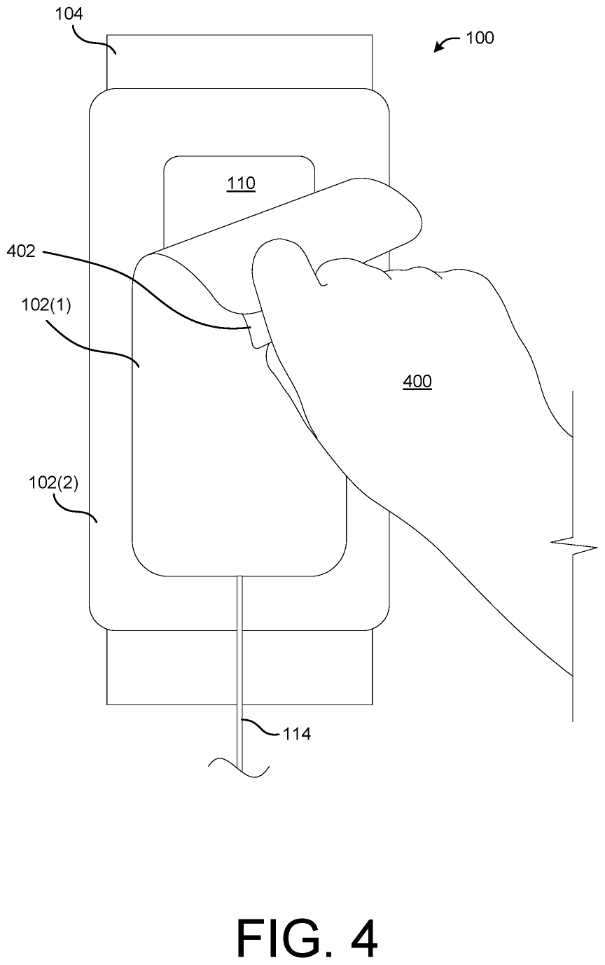

FIG. 4 illustrates the top view of the example electrode assembly 100 depicted in FIG. 1, and further depicting a user 400 removing the first electrode portion 102(1) from the second electrode portion 102(2). This illustrates a way to convert the electrode assembly 100 to an electrode having a reduced size. That is, the first electrode portion 102(1) is removable from the second electrode portion 102(2). In some examples, a stickiness of an adhesive that couples the second electrode portion 102(2) to the first electrode portion 102(1) is such that the force of gravity is not enough to separate the two electrode portions 102(1) and 102(2) from each other. Nevertheless, a user 400 can remove the first electrode portion 102(1) from the second electrode portion 102(2) with minimal effort notwithstanding the adhesive coupling between the electrode portions 102. In some examples, this removal with minimal effort is facilitated with a liner (e.g., plastic liner) that is interposed between the electrode portions 102 where the electrode portions 102 are in contact with each other. In this way, the first electrode portion 102(1) is configured to be used without the second electrode portion 102(2) to deliver therapy (e.g., defibrillation therapy) to a patient who is less than a threshold size and/or age, such as a pediatric patient. In the example of FIG. 4, the first electrode portion 102(1) is removable from the second electrode portion 102(2) by peeling the first electrode portion 102(1) away from the second electrode portion 102(2). In some examples, the first electrode portion 102(1) includes a pull tab 402 to aid the user 400 with peeling the first electrode portion 102(1) away from the second electrode portion 102(2). In some examples, the pull tab 402 is disposed at a corner 108 of the first electrode portion 102(1). In general, the pull tab 402 is configured to be grasped by the user 400 to peel the first electrode portion 102(1) away from the second electrode portion 102(2). In other examples, the corner 108 itself can be peeled up and grasped by the user 400. In these examples, the corner 108 is devoid of adhesive on the bottom surface to facilitate peeling the corner 108 up to grasp the corner 108 as a pull tab. In another example, a pull tab (e.g., similar to the pull tab 402) is included on multiple electrode portions 102, such a first pull tab on the first electrode portion 102(1) and a second pull tab on the second electrode portion 102(2). In this example, the pull tabs may have instructions or guidance (e.g., text, illustrations, symbols, and/or diagrams, etc.) printed thereon. The instructions or guidance printed on the respective pull tabs may be indicative of different sizes of patients, such as an adult patient, a pediatric patient, etc. The instructions or guidance printed on the pull tabs may help a user 400 quickly determine which pull tab to grasp and pull in order to remove the appropriate electrode portion(s) 102 for treating a given patient.

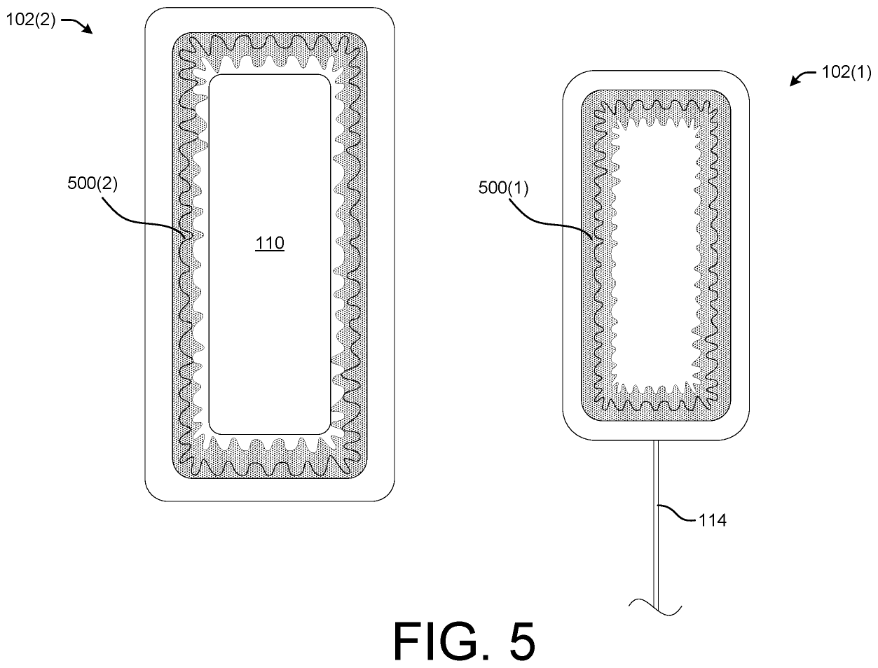

FIG. 5 illustrates a bottom view of the two electrode portions 102(1) and 102(2) of the electrode assembly 100 depicted in FIG. 1. The two electrode portions 102(1) and 102(2) are detached from one another and positioned in a side-by-side arrangement in FIG. 5. FIG. 5 also illustrates example conductive areas 500 of the electrode portions 102. A first conductive area 500(1) of the first electrode portion 102(1) is disposed on a bottom of the first electrode portion 102(1). A second conductive area 500(2) of the second electrode portion 102(2) is disposed on a bottom of the second electrode portion 102(2). These conductive areas 500 are sometimes referred to herein as “active areas 500” of the electrode portions 102. This is because therapy is delivered to the patient via the conductive areas 500 of the electrode portions 102. When the first electrode portion 102(1) is used without the second electrode portion 102(2), therapy (e.g., defibrillation therapy, such as an electrical shock) is delivered to a patient via the first conductive area 500(1) based on an electrical signal received by the first electrode portion 102(1) from a medical device (e.g., an external defibrillator) via the electrical cable 114. In this example, the patient is likely to be less than a threshold size and/or age, such as a pediatric patient. When the first electrode portion 102(1) is used together with the second electrode portion 102(2) (e.g., when the electrode assembly 100 is assembled, but the liner 104 is removed), therapy (e.g., defibrillation therapy, such as an electrical shock) is delivered to a patient via the first conductive area 500(1) and via the second conductive area 500(2) based on an electrical signal received by the first electrode portion 102(1) from a medical device (e.g., an external defibrillator) via the electrical cable 114. In this example, the patient is likely to be greater than a threshold size and/or age, such as an adult patient.

In some examples, the first conductive area 500(1) is proximate to and extends along the outer edge(s) 106 of the first electrode portion 102(1). In some examples, a central part of the first electrode portion 102(1) is devoid of the first conductive area 500(1), and a periphery (e.g., the extreme periphery) of the first electrode portion 102(1) is also devoid of the first conductive area 500(1). Said another way, the first conductive area 500(1), in some examples, takes the form of a strip(s) of electrically conductive material (sometimes shortened herein to “conductive material”) that substantially follows the outer edges 106 of the first electrode portion 102(1), but is inset from the outer edge(s) 106 to allow for adhesive at the periphery (e.g., the extreme periphery) of the first electrode portion 102(1). In other examples, the first conductive area 500(1) is a solid, contiguous area of conductive material that is positioned substantially at a center of the first electrode portion 102(1).

In some examples, the second conductive area 500(2) is proximate to and extends along the outer edge(s) 106 of the second electrode portion 102(2). In some examples, a periphery (e.g., the extreme periphery) of the second electrode portion 102(2) is devoid of the second conductive area 500(2). Because a cutout 110 is defined in the second electrode portion 102(2), there is no conductive area in the central part of the second electrode portion 102(2); the central part of the second electrode portion 102(2) (i.e., the cutout 110) is devoid of material whatsoever. In any case, the second conductive area 500(2), in some examples, is in the form of a strip(s) of conductive material that substantially follows the outer edge(s) 106 of the second electrode portion 102(2), but is inset from the outer edge(s) 106 to allow for adhesive at the periphery (e.g., the extreme periphery) of the second electrode portion 102(2). The second conductive area 500(2) also extends along and surrounds the inner edge(s) 112 of the second electrode portion 102(2).

In some examples, the conductive material of the conductive areas 500 is a metal, such as a tin plate. In some examples, the conductive material of the conductive areas 500 is silver-silver chloride. In this example, a silver-based conductive ink sits in a gel that includes a salt solution of silver chloride. In some examples, the individual conductive areas 500 include multiple, alternating layers of conductive ink and insulating ink. Such ink may be printed, layer-by-layer, to obtain the multiple layers of ink. This design of the conductive areas 500 lowers impedance, which lowers noise in the electrical signal received from the electrode assembly 100 when the electrode assembly 100 is used to monitor a physiological parameter of the patient. For example, the electrode assembly 100, or a portion(s) 102 thereof, may be used as an electrocardiogram (ECG) electrode to provide an ECG signal. The example design of the conductive area 500 reduces the noise in such an ECG signal. The example design of the conductive area 500 also increases the area over which to spread the electrical current, such as when delivering defibrillation therapy (e.g., an electrical shock), which reduces current density and hence electrical burns.

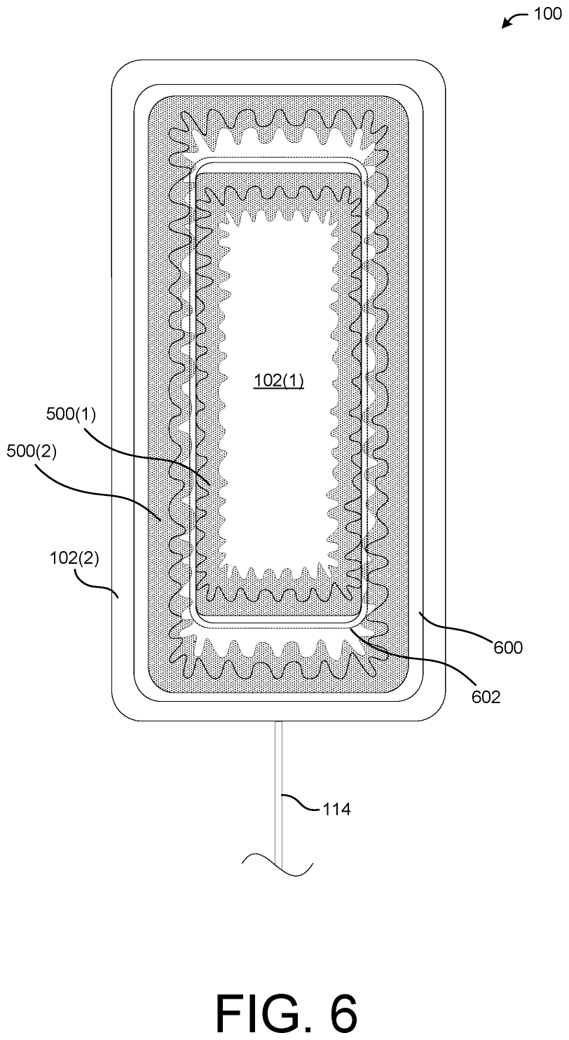

FIG. 6 illustrates a bottom view of the electrode assembly 100 depicted in FIG. 1 without the liner 104, and further depicting a perforated layer of gel 600 disposed on at least the respective conductive areas 500 of the electrode portions 102. In some examples, the gel 600 is an electrically-conductive material. In some examples, the gel 600 is a dielectric material. In some examples, the gel 600 has a relatively low impedance. In some examples, the gel 600 is a wet gel. In other examples, the gel 600 is a solid gel that is less aqueous than a wet gel. In the example of FIG. 6, the layer of gel 600 spans both conductive areas 500(1) and 500(2) such that the layer of gel 600 is disposed on, and covers, both conductive areas 500(1) and 500(2). This conductive area coverage of the gel 600 facilitates contact of the conductive areas 500(1) and 500(2) with the skin of the patient due to the adhesive properties of the gel 600.

The layer of gel 600 is also perforated by a perforation 602 in the layer of gel 600 that is substantially aligned with the cutout 110 defined in the second electrode portion 102(2). The perforation 602 in the layer of gel 600 facilitates separating the electrode portions 102 from each other, such as removing (e.g., peeling or tearing away) the first electrode portion 102(1) from the second electrode portion 102(2). When the electrode assembly 100 is assembled and the electrode portions 102(1) and 102(2) are coupled together, the layer of gel 600 also provides electrical conductivity between the first electrode portion 102(1) and the second electrode portion 102(2). This is why an electrical cable is not coupled to the second electrode portion 102(2). Instead, the electrical cable 114 that is coupled to the first electrode portion 102(1) is sufficient for delivering therapy (e.g., defibrillation therapy) via both conductive areas 500(1) and 500(2) to a patient who is greater than a threshold size and/or age, such as an adult patient. In other words, electrical signals from a medical device (e.g., an external defibrillator) can travel from the first conductive area 500(1) to the second conductive area 500(2), and electrical signals can also travel in the opposite direction between the conductive areas 500, via the layer of gel 600 that covers, and provides electrical conductivity between, both conductive areas 500(1) and 500(2). In other examples, a separate electrical cable, such as an additional electrical cable(s) similar to the electrical cable 114, is coupled to each additional electrode portion 102 of the electrode assembly 100 to facilitate delivery of therapy (e.g., an electrical shock) via the plurality of conductive areas 500.

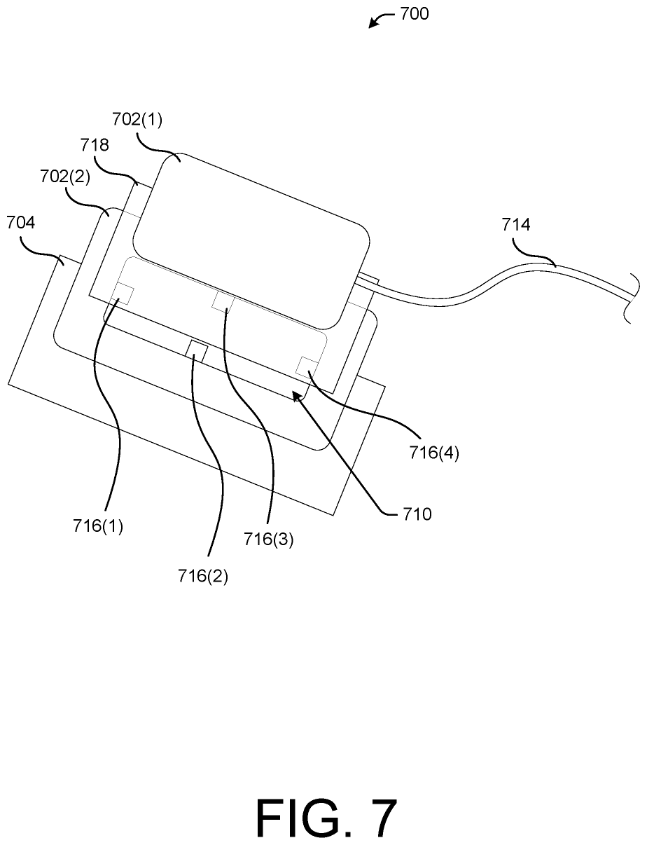

FIG. 7 illustrates a perspective exploded view of another example electrode assembly 700 having two electrode portions 702(1) and 702(2), the example electrode assembly 700 further including conductive elements 716(1), 716(2), 716(3), and 716(4) (collectively 716), and a conductive film 718. The two electrode portions 702(1) and 702(2) may be similar to the electrode portions 102(1) and 102(2), respectively. Furthermore, the liner 704 and the electrical cable 714 may be similar to the liner 104 and the electrical cable 114, respectively.

In the example of FIG. 7, the conductive elements 716 (sometimes referred to herein as “conductive tabs 716”) are distributed (e.g., evenly distributed) about the inner edge(s) 112 of the second electrode portion 702(2). The conductive elements 716 are made of conductive material, such as metal, a printed ink, etc. When the electrode assembly 700 is assembled, the electrode portions 702 are coupled together such that the conductive elements 716 provide respective electrical bridges between the respective conductive areas of the electrode portions 702. That is, the conductive elements 716 couple the first conductive area of the first electrode portion 702(1) to the second conductive area of the second electrode portion 702(2). This creates a stable electrical connection between the conductive areas of the respective electrode portions 702 that may improve the electrical conductivity between the electrode portions 702, as compared to the layer of gel 600 exclusively acting as the electrical bridge between the respective conductive areas of the electrode portions 702, without the use of the conductive elements 716. The conductive elements 716 may also facilitate separating the electrode portions 702 from each other, such as removing the first electrode portion 702(1) from the second electrode portion 702(2). Although four example square-shaped conductive elements 716 are depicted in FIG. 7, it is to be appreciated that the electrode assembly 700 can include any suitable shape and number of conductive elements 716, such as a single conductive element 716, two conductive elements 716, three conductive elements 716, or more than four conductive elements 716.

The conductive film 718 (sometimes referred to herein as a “conductive film separation layer 718”) of the electrode assembly 700 is made of a conductive material, such as a metal foil, which further acts as an electrical bridge between the respective conductive areas of the electrode portions 702. In the example of FIG. 7, a first layer of gel (not shown in FIG. 7) is disposed on a first conductive area (not shown in FIG. 7) of the first electrode portion 702(1). The first conductive area of the first electrode portion 702(1) may be similar to the first conductive area 500(1) depicted in FIG. 5. In some examples, the layer of gel disposed on the first conductive area covers the center part of the first electrode portion 702(1) that is potentially devoid of conductive material. Covering the center (non-conductive) part of the first electrode portion 702(1) with a gel layer reduces the complexity of manufacturing the electrode assembly 700 and/or improves adhesion of the conductive film 718 to the bottom of the first electrode portion 702(1). Continuing with reference to FIG. 7, the conductive film 718, in this example, is disposed on the first layer of gel of the first electrode portion 702(1), and a second layer of gel (not shown in FIG. 7) is disposed on both the second conductive area of the second electrode portion 702(2) and the conductive film 718. In some examples, the second layer of gel is applied over the entire bottom surface of the electrode assembly 700, except the periphery of the second electrode portion 702(2). The second conductive area of the second electrode portion 702(2) may be similar to the second conductive area 500(2) depicted in FIG. 5. Accordingly, the second layer of gel may cover the center part of the conductive film 718 that is exposed through the cutout 710 of the second electrode portion 702(2) when the electrode assembly 700 is assembled. In this configuration, when the first electrode portion 702(1) is removed from the second electrode portion 702(2), a layer of gel remains in the cutout 710 of the second electrode portion 702(2) because the second layer of gel is disposed, in part, on a center part of the conductive film 718.

Comparing the examples of FIGS. 6 and 7, the example of FIG. 6—which is devoid of conductive elements 716 and conductive film 718—reduces the quantity of gel material that is used to cover at least the conductive areas on the bottom of each electrode portion 102, which translates into cost savings over a large manufacturing volume. The example of FIG. 6 also reduces the number of steps to make the electrode assembly 100 during manufacturing. For example, to make the electrode assembly 700 of FIG. 7, two different layers of gel are applied, with a conductive film 718 separating the two gel layers, and the first layer of gel has to cure before applying the second layer of gel. The manufacturing process to make the electrode assembly 100 illustrated in FIG. 6, for example, reduces the multiple gel-applying steps into a single gel-applying step where one layer of gel 600 is applied to a bottom of the second electrode portion 102(2) and to a bottom of the first electrode portion 102(1), which is exposed through the cutout 110 of the second electrode portion 102(2). An advantage of the example configuration shown in FIG. 7 is that the conductive element(s) 716 and conductive film 718 aid in the separation of the electrode portions 702 from one another, such as making it easier for a user to remove the first electrode portion 702(1) from the second electrode portion 702(2), as compared to the perforated gel layer 600 configuration depicted in FIG. 6. That said, the perforation 602 in the layer of gel 600 is configured to facilitate the separation of the electrode portions 102. Additionally, or alternatively, the example configuration shown in FIG. 7 may aid in reattaching (or reassembling) a removed electrode portion(s) 702 to the remaining electrode portion(s) 702. For example, if a user 400 removed the first electrode portion 702(1) by mistake, the user 400 can reattach the first electrode portion 702(1) to the second electrode portion 702(2), and the reassembled electrode assembly 700 may exhibit sufficient electrical conductivity between the respective electrode portions 702.

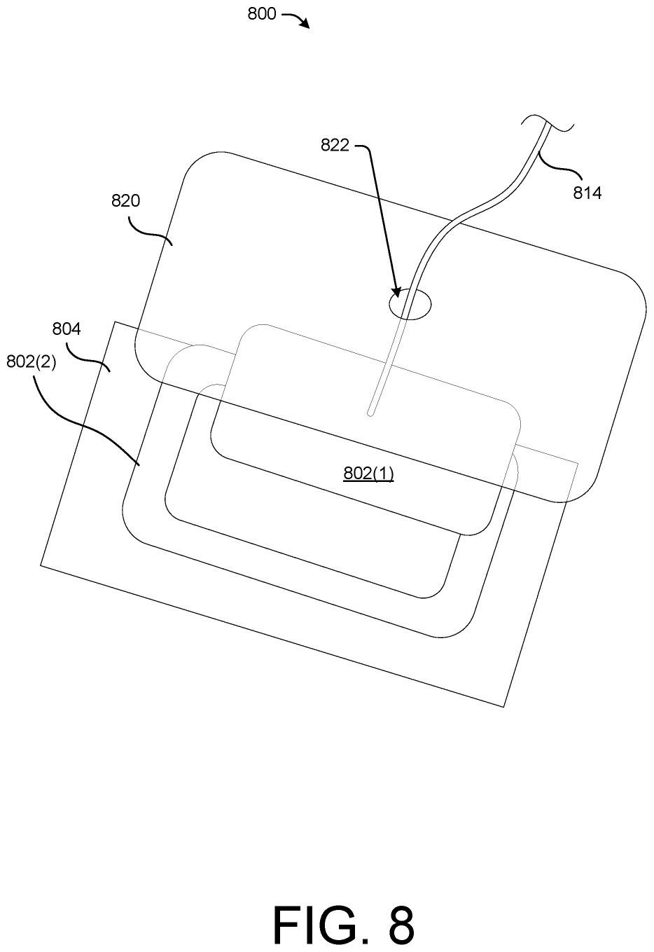

FIG. 8 illustrates a perspective exploded view of yet another example electrode assembly 800 having two electrode portions 802(1) and 802(2). The two electrode portions 802(1) and 802(2) may be similar to the electrode portions 102(1) and 102(2), respectively. Furthermore, the liner 804 and the electrical cable 814 may be similar to the liner 104 and the electrical cable 114, respectively.

The example electrode assembly 800 further includes an adhesive backing 820. In some examples, the adhesive backing 820 (sometimes referred to herein as “adhesive tape 820”) spans the multiple electrode portions 802 and is disposed on a topmost electrode portion 802, which, in the example of FIG. 8 is the first electrode portion 802(1). The adhesive backing 820 is depicted as a transparent sheet, but it is to be appreciated that the adhesive backing 820 is opaque or semi-transparent in other examples. The adhesive backing 820 reinforces the coupling (e.g., adhesion) between the multiple electrode portions 802 to prevent the electrode portions 802 from inadvertently separating during use of the electrode assembly 800. If a user wants to remove the first electrode portion 802(1) from the second electrode portion 802(2), the user first removes the adhesive backing 820, such as by peeling the adhesive backing 820 away from the remainder of the electrode assembly 800, and then the user removes the first electrode portion 802(1) from the second electrode portion 802(2).

Furthermore, in the example of FIG. 8, the adhesive backing 820 includes an aperture 822 at or near a center of the adhesive backing 820, and the electrical cable 814 is coupled to the first electrode portion 802(1) at a center of the first electrode portion 802(1). The electrical cable 814 extends from the center of the first electrode portion 802(1), passes through the aperture 822, and is coupled to a medical device (e.g., an external defibrillator) at the other end of the electrical cable 814. This configuration of the electrical cable 814 allows for improved contact between the adhesive backing 820 and particular electrode portions 802, such as the second electrode portion 802(2). By contrast, in the example of FIG. 1, the electrical cable 114 is coupled to the first electrode portion 102(1) at an edge 106 of the first electrode portion 102(1).

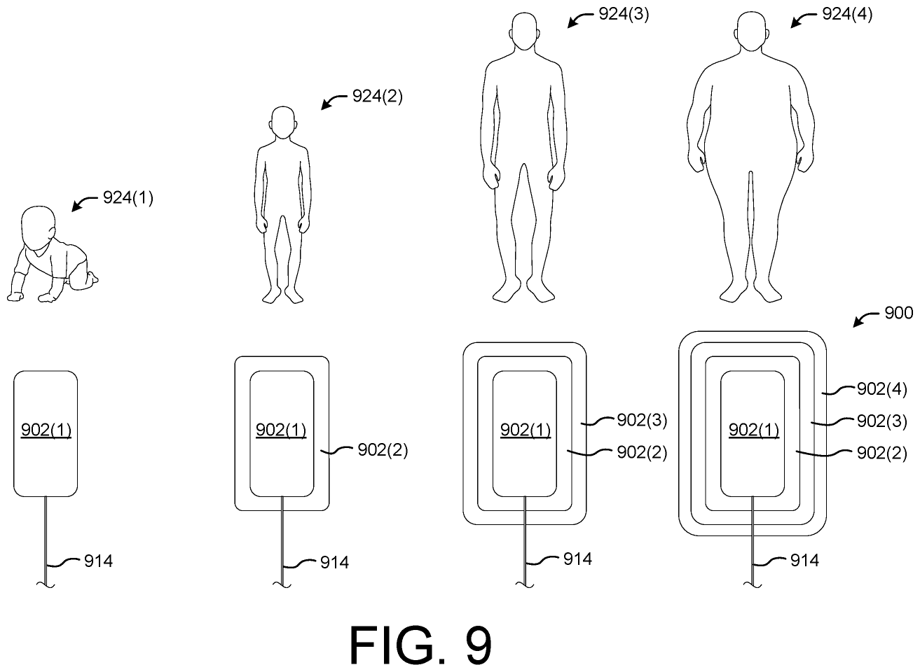

FIG. 9 illustrates another example electrode assembly 900 having four electrode portions 902(1), 902(2), 902(3), and 902(4) (collectively 902), each electrode portion 902 being removable to accommodate a variety of different-sized patients 924. The first electrode portion 902(1) may be similar to the first electrode portion 102(1). Furthermore, the second electrode portion 902(2), the third electrode portion 902(3), and the fourth electrode portion 902(4) may be similar to the second electrode portion 102(2) described herein. It is to be appreciated, however, that the third electrode portion 902(3) is larger than the second electrode portion 902(2), and that the fourth electrode portion 902(4) is larger than the third electrode portion 902(3). For example, the second electrode portion 902(2) may have a first cutout similar to the cutout 110 described herein, and the third electrode portion 902(3) may have a second cutout similar to, but larger than, the first cutout 110 of the second electrode portion 902(2). Thus, when the third electrode portion 902(3) is disposed on the second electrode portion 902(2) at a second edge(s) 106 of the second electrode portion 902(2), the first electrode portion 902(1) and the second electrode portion 902(2) span the second cutout of the third electrode portion 902(3). Similarly, the fourth electrode portion 902(4) may have a third cutout similar to, but larger than, the second cutout of the third electrode portion 902(3). Thus, when the fourth electrode portion 902(4) is disposed on the third electrode portion 902(3) at a third edge(s) 106 of the third electrode portion 902(3), the first electrode portion 902(1), the second electrode portion 902(2), and the third electrode portion 902(3) span the third cutout of the fourth electrode portion 902(4). It is to be appreciated that an electrode assembly can have any suitable number of electrode portions 102, 702, 802, 902, such as two electrode portions, three electrode portions, or more than four electrode portions. Moreover, the arrangement of electrode portions can be similar to the various arrangements disclosed herein, by way of example. The example electrode assembly 900 depicted in FIG. 9 that includes four electrode portions 902 is merely an example where the number of electrode portions 902 is equal to four.

A user can remove any number of electrode portions 902 from the others in order to adapt the electrode assembly 900 to a size that is suitable for the size of the patient 924. FIG. 9 illustrates four different types of patients 924, as an illustrative example: an infant patient 924(1), a pediatric patient 924(2), an adult patient 924(3), and a large adult patient 924(4). The delineation between these different types of patients 924(1)-(4) may be established using any suitable metric or combination of metrics including size (e.g., height, weight, demi-span, and/or any other suitable size metric) and/or age. For example, an infant patient 924(1) may be defined as any patient less than a first threshold age (e.g., less than 2 years old) and/or less than a first threshold size (e.g., less than a first threshold weight and/or less than a first threshold height, etc.). Meanwhile, a pediatric patient 924(2) may be defined as any patient equal to or greater than the first threshold age and less than a second threshold age (e.g., less than 18 years old) and/or greater than the first threshold size and less than a second threshold size. The adult patient 924(3) and large adult patients 924(4) may be defined using similar size and/or age thresholds and/or ranges.

In the example of FIG. 9, for an infant patient 924(1), the first electrode portion 902(1) is removable from the second electrode portion 902(2), from the third electrode portion 902(3), and from the fourth electrode portion 902(4) and is configured to be used without the second electrode portion 902(2), without the third electrode portion 902(3), and without the fourth electrode portion 902(4) to deliver therapy to the infant patient 924(1), such as via the first conductive area of the first electrode portion 902(1). For a pediatric patient 924(2), the second electrode portion 902(2) is removable from the third electrode portion 902(3) and from the fourth electrode portion 902(4) and is configured to be used with the first electrode portion 902(1), without the third electrode portion 902(3), and without the fourth electrode portion 902(4) to deliver therapy to the pediatric patient 924(2), such as via the first and second conductive areas of the first and second electrode portions 902(1) and 902(2). For an adult patient 924(3) who is less than a threshold size, the third electrode portion 902(3) is removable from the fourth electrode portion 902(4) and is configured to be used with the first electrode portion 902(1), with the second electrode portion 902(2), and without the fourth electrode portion 902(4) to deliver therapy to the adult patient 924(3) who is less than a threshold size, such as via the first, second, and third conductive areas of the first, second, and third electrode portions 902(1)-(3). For an adult patient 924(4) who is greater than a threshold size, a user does not have to remove any of the electrode portions 902. Instead, the first electrode portion 902(1), the second electrode portion 902(2), the third electrode portion 902(3), and the fourth electrode portion 902(4) are configured to be used together to deliver therapy to the adult patient 924(4) who is greater than a threshold size, such as via the first, second, third, and fourth conductive areas of the first, second, third, and fourth electrode portions 902(1)-(4). Said another way, the electrode assembly 900 is usable as-is for delivering therapy to the large adult patient 924(4) who is greater than a threshold size, or a user can remove (e.g., peel away) the first, second, and third electrode portion 902(1)-(3) and use them together to deliver therapy to an adult patient 924(3) who is less than a threshold size, or the user can remove (e.g., peel away) the first and second electrode portions 902(1) and 902(2) and use them together to deliver therapy to the pediatric patient 924(2) (e.g., a child), or the user can remove (e.g., peel away) the first electrode portion 902(1) and use it by itself to deliver therapy to an infant patient 924(1).

Accordingly, the example electrode assembly 900 can accommodate a variety of different-sized patients ranging from infant 924(1) to large adult 924(4). This eliminates the need for users of medical devices (e.g., external defibrillators) to maintain a separate set of electrodes for each type of patient 924 depicted in FIG. 9. Additionally, or alternatively, a user can adapt the electrode assembly 900 to deliver therapy (e.g., defibrillation therapy) to a patient in a more appropriate way (e.g., through delivery of an electrical shock via an appropriately-sized electrode at an appropriate level of energy), as opposed to using a fixed-size electrode on the variety of different-sized patients 924 depicted in FIG. 9.

In some examples, on each electrode portion 902 there may be a unique visual indicator (e.g., text, a symbol, a color, a pattern, etc.) and/or information to indicate to a user which electrode portion(s) 902 is/are appropriate for the patient in question. For example, a range(s) of length dimensions and/or weight dimensions and/or ages may be printed on individual electrode portions 902. In an example, the fourth electrode portion 902(4) may have “Adults: 250 pounds (lbs) and above” printed thereon, while the third electrode portion 902(3) may have “Adults: 100-250 lbs” printed thereon, and so on and so forth for the other electrode portions 902 in order to indicate the type of patient (e.g., in terms of size and/or age of the patient) that corresponds to the particular electrode portion(s) 902 of the electrode assembly 900.

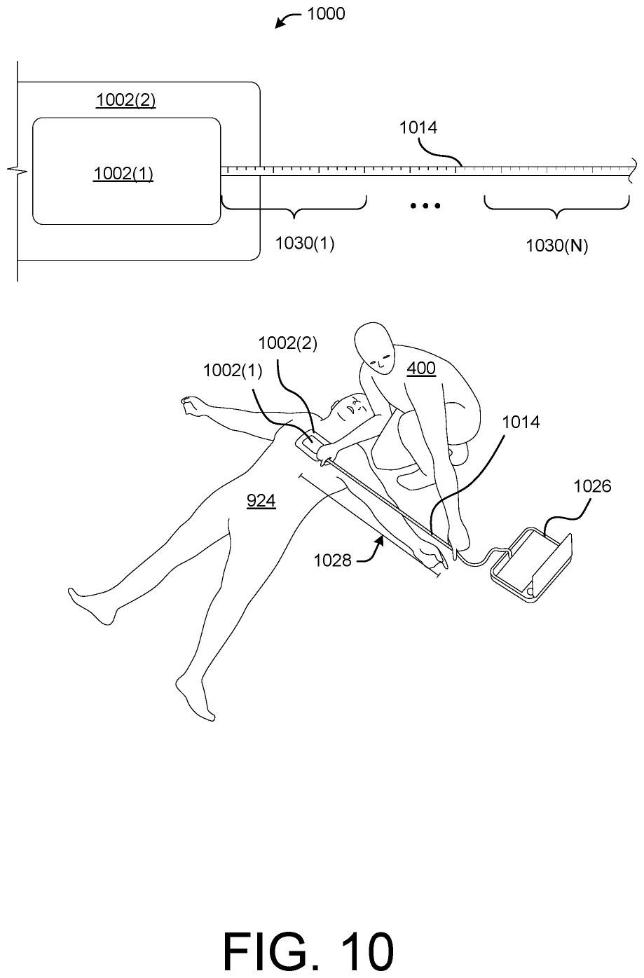

FIG. 10 illustrates an electrical cable 1014 of an electrode assembly 1000 having a measurement scale printed thereon, and a technique for determining which electrode portion(s) 1002 to use on a patient 924 based on a portion of the patient's body measured with the measurement scale. The electrode portions 1002(1) and 1002(2) may be similar to the electrode portions 102(1) and 102(2), respectively. Furthermore, the electrical cable 1014 may be similar to the electrical cable 114, at least to the extent that the electrical cable 1014 is configured to deliver electrical signals from a medical device to the first electrode portion 1002(1), or vice versa. As mentioned, a measurement scale is printed on the electrical cable 1014. The measurement scale may include evenly distributed markings along a length of the electrical cable 1014 that indicate a length measurement. The measurement scale can be implemented with any suitable unit of length measurement or any combination of different units of length, such as inches, centimeters, etc.

In an example, a user 400 of a medical device 1026 (e.g., an external defibrillator) measures a portion of a body of a patient 924 using the measurement scale printed on the electrical cable 1014 to obtain a measurement. In the example of FIG. 10, the portion of the patient's body is a demi-span 1028 of the patient's body. The demi-span 1028 is the distance from the middle of the sternal notch to the tip of the middle finger of an extended arm. In some examples, the electrical cable 1014 is about 1 meter (m) (3 feet (ft)) long, or longer, which is suitable for measuring the demi-span 1028 of infant patients 924(1) and pediatric patients 924(2), and of the average adult patient 924(3). It is to be appreciated, however, that the measurement scale of the electrical cable 1014 may be used to measure other parts of the patient's body, such as the patient's height (e.g., the distance from the bottom of the foot to the top of the head), and/or part of the patient's height (e.g., from the waist to the top of the head), and/or the patient's width, girth, etc. The electrical cable 1014 is also conveniently positioned proximate to the patient 924 prior to delivery of therapy via one or more of the electrode portions 1002, which makes the electrical cable 1014 a convenient measurement tool to use for determining the size of the patient 924.

Based on the measurement (e.g., the measurement of the demi-span 1028) obtained using the measurement scale of the electrical cable 1014, the user 400 may determine which electrode portion(s) 1002 correspond(s) to the size of the patient 924. The user 400 may then utilize the determined electrode portion(s) 1002 to deliver therapy (e.g., defibrillation therapy) to the patient 924. For example, the user 400 may, in his/her head, or using reference information, determine (e.g., estimate) the height of the patient 924 based on the measured demi-span 1028 of the patient 924, and the user 400 then determines which electrode portion(s) 1002 to use based on the determined height of the patient 924. In another example, the user 400 may, in his/her head, or using reference information, determine (e.g., estimate) the ideal body weight of the patient 924 based on the height of the patient, which may be determined (e.g., estimated) based on the measured demi-span 1028 of the patient 924, and the user 400 may then determine which electrode portion(s) 1002 to use based on the determined ideal body weight of the patient 924.

In some examples, the measurement scale printed on the electrical cable 1014 indicates length measurements in any suitable unit(s) of measurement, such as inches and/or centimeters, which can be used to measure a portion of the patient's body, such as the demi-span 1028. In other examples, the measurement scale printed on the electrical cable 1014 indicates height measurements of the patient 924 for a given demi-span 1028 length measurement. This allows the user 400 to quickly determine the height of the patient 924 by measuring the demi-span 1028 of the patient 924. In other examples, the measurement scale printed on the electrical cable 1014 indicates ideal body weight measurements of the patient 924 for a given demi-span 1028 length measurement. This example measurement scale may include separate scales for male and female patients whose ideal body weight may be calculated differently based on length measurement of a body portion, such as the demi-span 1028. This allows the user 400 to quickly determine the ideal body weight of the patient 924 by measuring the demi-span 1028 of the patient 924. In other examples, multiple electrical cables including the electrical cable 1014 each have a different measurement scale printed thereon, and the user 400 selects the electrical cable 1014 that is most useful to them.

In some examples, different portions 1030(1) to 1030(N) (or sections) of the measurement scale printed on the electrical cable 1014 are color-coded with different colors that correspond to different colors of the electrode portions 1002. For example, the electrode portions 902 of the electrode assembly 900 may be color-coded with different colors, such as an orange first electrode portion 902(1), a red second electrode portion 902(2), a green third electrode portion 903(3), and a blue fourth electrode portion 902(4). With this color-coding scheme, the measurement scale printed on the electrical cable 914 includes four portions 1030 (or sections) including a first portion 1030 at an end of the electrical cable 914 that is orange, a second portion 1030 adjacent to the first portion that is red, a third portion 1030 adjacent the second portion that is green, and a fourth portion 1030 adjacent the third portion that is blue. Thus, when the user 400 measures a body portion (e.g., the demi-span 1028) of the patient 924, the user 400 can quickly determine that the body portion is within a portion 1030 of the measurement scale of the electrical cable 914 that is color-coded with a particular color (e.g., green), and then the user 400 determines that the third electrode portion 902(3) is also green, which matches the color of the portion 1030 of the measurement scale corresponding to the measured body portion (e.g., demi-span 1028) of the patient 924. In this manner, the user 400 removes the third electrode portion 902(3) from the fourth electrode portion 904(4) (e.g., by peeling the third electrode portion 902(3) away from the fourth electrode portion 904(4)), and uses the first, second, and third electrode portions 902(1)-(3) together to deliver therapy (e.g., defibrillation therapy) to the patient 924, who may be an adult patient based on the demi-span 1028 measurement falling within the green portion 1030 of the measurement scale.

In some examples, the user 400 determines to use the entire electrode assembly 1000 on the patient 924 depicted in FIG. 10. In these examples, the user 400 places the electrode assembly 1000 on a patient 924, and causes delivery of therapy to the patient via the respective conductive areas of the electrode portions 1002, such as by operating the medical device 1026 (e.g., an external defibrillator).

In some examples, the measurement (e.g., the measurement of the demi-span 1028) obtained using the measurement scale of the electrical cable 1014 is used in other ways. For instance, the user 400 may input the measurement into the medical device 1026 (e.g., a monitor-defibrillator), such as by providing user input (e.g., typing, speaking, etc.) to the medical device 1026, and the medical device 1026 may automatically switch to a particular usage mode of a plurality of usage modes for delivering therapy to the patient 924 at an appropriate energy level based on the measurement, to monitor physiological parameters of the patient 924 and output an alarm if a physiological parameter is anomalous based on threshold associated with the measurement, to providing information, such as charts, tables, etc., of drug dosing based on the measurement, and/or to provide other functionality via the medical device 1026. In an example, the measurement that the user 400 inputs to the medical device 1026 corresponds to a pediatric patient, and the medical device 1026 (e.g., a monitor-defibrillator) automatically switches to a pediatric energy level to deliver an electrical shock to the patient, monitors a physiological parameter(s) and compares the parameter data to a pediatric threshold(s), and/or displays charts, tables, etc. on a display of the medical device 1026 to provide pediatric-specific information to the user 400.

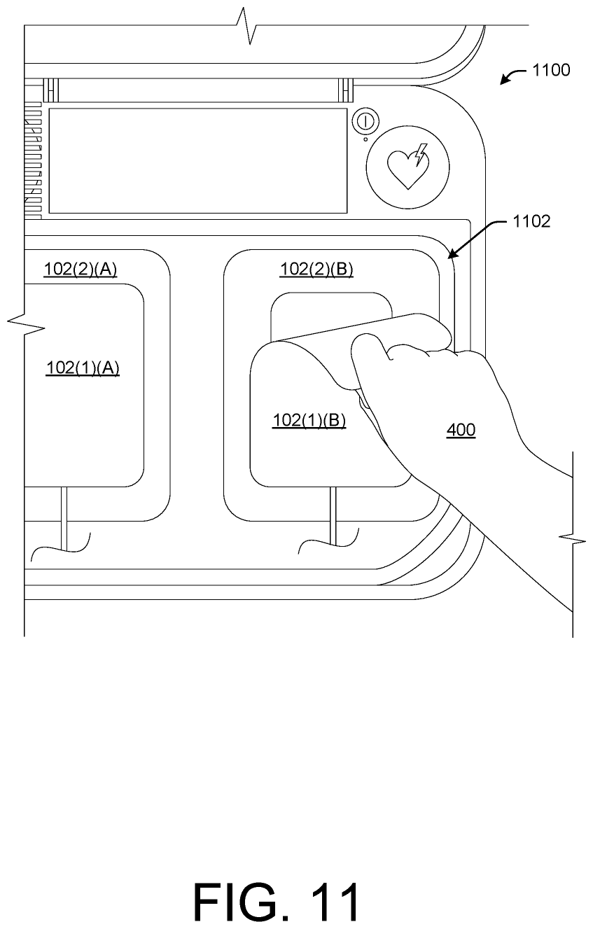

FIG. 11 illustrates a set of electrode assemblies disposed in an electrode storage tray 1102 of an external defibrillator 1100, and further depicts a user 400 removing a first electrode portion 102(1)(B) of one electrode assembly from the electrode storage tray 1102. Each example electrode assembly depicted in FIG. 11 is a two-portion electrode assembly having a first electrode portion 102(1)(A), 102(1)(B) and a second electrode portion 102(2)(A), 102(2)(B). Accordingly, the first electrode portion 102(1)(A), 102(1)(B) may be usable—without the second electrode portion 102(2)(A), 102(2)(B)—to provide defibrillation therapy to a pediatric patient 924(2), for example. In one example, after observing the size and/or age of the patient 924(2), and/or after determining the size of the patient 924(2) by measuring a portion of a body of the patient 924(2), such as the demi-span 1028 of the patient 924(2), the user 400 determines that the first electrode portion 102(1)(A), 102(1)(B) corresponds to the size of the patient 924(2). Upon making this determination, the user 400 removes the first electrode portions 102(1)(A), 102(1)(B) from the second electrode portion 102(2)(A), 102(2)(B), respectively. In the example of FIG. 11, the user 400 is in the process of removing the first electrode portion 102(1)(B) from the electrode storage tray 1102, such as by peeling the first electrode portion 102(1)(B) away from the second electrode portion 102(2)(B) and leaving the second electrode portion 102(2)(B) in the electrode storage tray 1102. In this way, the user 400 is able to place the first electrode portions 102(1)(A), 102(1)(B) on the patient 924(2) and cause delivery of defibrillation therapy to the patient 924(2) via the first conductive areas (e.g., 500(1)) of the first electrode portion 102(1)(A), 102(1)(B). For example, the user 400 operates the external defibrillator 1100 to cause an electrical shock to be delivered to the patient 924(2) via the first conductive areas (e.g., 500(1)) of the first electrode portions 102(1)(A), 102(1)(B). Accordingly, the user 400 is able to conveniently select the appropriate electrode portion(s) 102 to use by removing that/those portion(s) 102 from the electrode storage tray 1102, possibly without removing the entire electrode assembly 100 from the electrode storage tray 1102, as depicted in the example of FIG. 11.

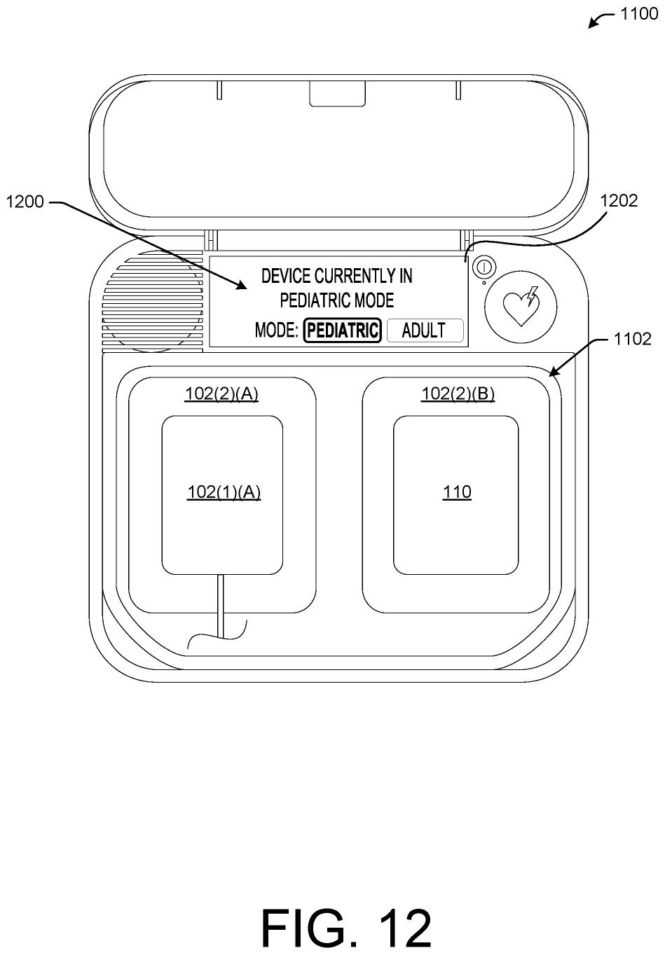

FIG. 12 illustrates the example external defibrillator 1100 depicted in FIG. 11 while outputting a notification 1200 that the external defibrillator 1100 is in a pediatric mode. In some examples, the external defibrillator 1100 is configured to determine whether one or more electrode portions 102 have been removed from the electrode storage tray 1102, as well as which electrode portion(s) 102 has/have been removed. In some examples, the external defibrillator 1100 includes one or more sensors configured to sense a parameter(s) indicative of a presence or an absence of an electrode portion(s) 102 within the electrode storage tray 1102, and the external defibrillator 1100 includes logic to determine, based on the sensed parameter(s), whether an electrode portion(s) 102 has/have been removed from the electrode storage tray 1102, and which electrode portion(s) 102 has/have been removed. Furthermore, depending on which electrode portion(s) 102 is/are removed, the external defibrillator 1100 is configured to select (e.g., automatically, without user intervention) a particular usage mode of a plurality of usage modes in which the external defibrillator 1100 is to operate. In an example, the plurality of usage modes include two usage modes: an adult mode and a pediatric mode. In another example, the plurality of usage modes include three usage modes: an adult mode, a pediatric mode, and an infant mode. In yet another example, the plurality of usage modes include four usage modes: a large adult mode, an adult mode, a pediatric mode, and an infant mode. The number and types of usage modes described herein are merely exemplary.

In the example of FIG. 12, consider an external defibrillator 1100 that is configured to operate in one of two usage modes: a pediatric mode, or an adult mode. Accordingly, when the user 400 removes first electrode portion 102(1)(B) from the electrode storage tray 1102, the external defibrillator 1100 (e.g., via one or more sensors), determines that the first electrode portion 102(1)(B) has been removed from the electrode storage tray 1102 and/or that the second electrode portion 102(2)(B) remains disposed in the electrode storage tray 1102. The user 400 also removes the other first electrode portion 102(1)(A) from the electrode storage tray 1102, and a similar determination is made by the external defibrillator 1100. Based on the first electrode portion 102(1)(B) (and possibly the first electrode portion 102(1)(A)) having been removed from the electrode storage tray 1102 and/or based on the second electrode portion 102(2)(B) (and possibly the second electrode portion 102(2)(A)) remaining disposed in the electrode storage tray 1102, the external defibrillator 1100 selects (e.g., automatically, without user intervention) the pediatric mode in which the external defibrillator 1100 is to operate for delivering defibrillation therapy to a patient 924. In some examples, the external defibrillator 1100 is configured to select (e.g., automatically, without user intervention), based on the selected usage mode, an energy level at which to deliver an electrical shock via the removed electrode portion(s) 102. This energy level is sometimes referred to herein as the “defibrillation energy.” Continuing with the running example, the external defibrillator 1100 selects, based on the selected pediatric mode, an energy level at which to deliver an electrical shock via the first electrode portions 102(1)(A), 102(1)(B), and this selected energy level is suitable for a pediatric patient 924(2), for example, which is lower than an energy level used to defibrillate an adult patient 924(3), for example.

Furthermore, the external defibrillator 1100 in FIG. 12 is shown as outputting, via a display 1202 of the external defibrillator 1100, a notification 1200 that the external defibrillator 1100 is in the pediatric mode. This is to inform the user 400 of the currently-selected usage mode so that the user 400 may determine whether the selected usage mode is correct for the size and/or age of the patient 924, or so that the user 400 can determine whether to override the selected usage mode for another reason. It is to be appreciated that a notification such as the notification 1200 may be output via any suitable output device of the external defibrillator 1100, such as the display 1202, a speaker(s), an indicator light(s) (e.g., a light emitting diode(s) (LEDs)), and/or a haptic mechanism, etc.

In some examples, the display 1202 may be a touch-sensitive display 1202 that functions as both an output device and an input device of the external defibrillator 1100. Accordingly, the user 400 is able to provide user input to the display 1202 to select a usage mode. For example, the user 400 may touch “ADULT” on the display 1202 to change the usage mode from pediatric mode to adult mode. After the external defibrillator 1100 receives, via the display 1202, the user input to select the adult mode, the external defibrillator 1100 changes the notification 1200 presented on the display 1202 to indicate that the external defibrillator 1100 is in the adult mode, such as by presenting “ADULT” in bold typeface, and no longer presenting “PEDIATRIC” in bold typeface. In other words, the user 400 is able to override the device-selected usage mode selected by the external defibrillator 1100. It is to be appreciated that user input may be received via any suitable input device of the external defibrillator 1100, such as the touch-sensitive display 1202, a microphone(s), a button(s), a keyboard(s)/keypad(s), a mouse/pointer, and/or a rotating dial, etc.

In some examples, the external defibrillator 1100 is configured to request (e.g., automatically, without user intervention), based on the selected usage mode, the user 400 to specify an estimated patient age by providing user input (e.g., typing, speaking, etc.) to the external defibrillator 1100. The user-specified patient age can then be used to automatically set or adjust the functionality of the external defibrillator 1100. For example, besides, or in addition to, setting the energy level for providing defibrillation therapy, the external defibrillator 1100 may monitor physiological parameters of the patient 924 and output an alarm if a physiological parameter is anomalous based on threshold associated with the user-specified patient age, provide information, such as charts, tables, etc., of drug dosing based on the user-specified patient age, and/or to provide other functionality via the external defibrillator 1100.

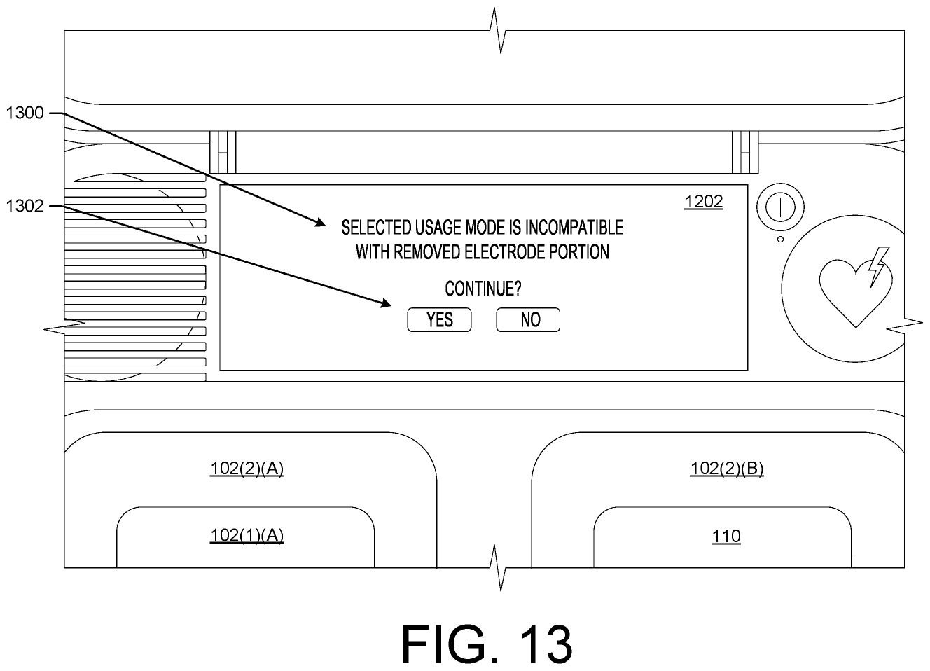

FIG. 13 illustrates a close-up view of the display 1202 of the example external defibrillator 1100 depicted in FIG. 11. The display 1202 in FIG. 13 is outputting an indication 1300 that a user-selected mode is incompatible with the removed electrode portion(s) 102, and a request 1302 for the user 400 to acknowledge the incompatibility. Consider an example where the user 400 removes the first electrode portion 102(1)(B) (and possibly the other first electrode portion 102(1)(A)) from the electrode storage tray 1102, and the pediatric mode is automatically selected by the external defibrillator 1100 based on the detected removal of the electrode portion(s) 102, and then the user 400 subsequently overrides the device-selected pediatric mode by providing user input to change the usage mode of the external defibrillator 1100 to the adult mode. In this example, the display 1202 (and/or another output device(s) of the external defibrillator 1100) outputs the indication 1300 to indicate to the user 400 that the user-selected adult mode is incompatible with the first electrode portion 102(1)(B) (and possibly the other first electrode portion 102(1)(A)) that has been removed from the electrode storage tray 1102. If the user 400 inadvertently changed the usage mode to the adult mode, then this indication 1300 may provide the user 400 with an opportunity to change the usage mode back to the pediatric mode. Otherwise, if the user 400 intended to change the usage mode to the adult mode in this example, the user 400 may provide user input to acknowledge that the adult mode is incompatible with the first electrode portion 102(1)(B) (and possibly the other first electrode portion 102(1)(A)) that has been removed from the electrode storage tray 1102. This gives a trained user 400 the ability to override a device-selected user mode and continue using the external defibrillator 1100 despite the incompatibility, if, say, the user 400 determines it is safe and/or effective to do so. In some examples, the external defibrillator 1100 may not be operable to deliver defibrillation therapy via the removed electrode portion(s) 102 until the external defibrillator 1100 receives, via the touch-sensitive display 1202 (and/or another input device of the external defibrillator 1100), user input acknowledging the incompatibility of the user-selected usage mode and the removed electrode portion(s) 102. In some examples, the external defibrillator 1100 may not be operable to deliver defibrillation therapy via the removed electrode portion(s) 102 until the user 400 authenticates as an authorized (e.g., trained) user 400, such as by inputting credentials, multi-factor authentication (MFA), providing biometric information (e.g., fingerprint, iris scan, etc.).

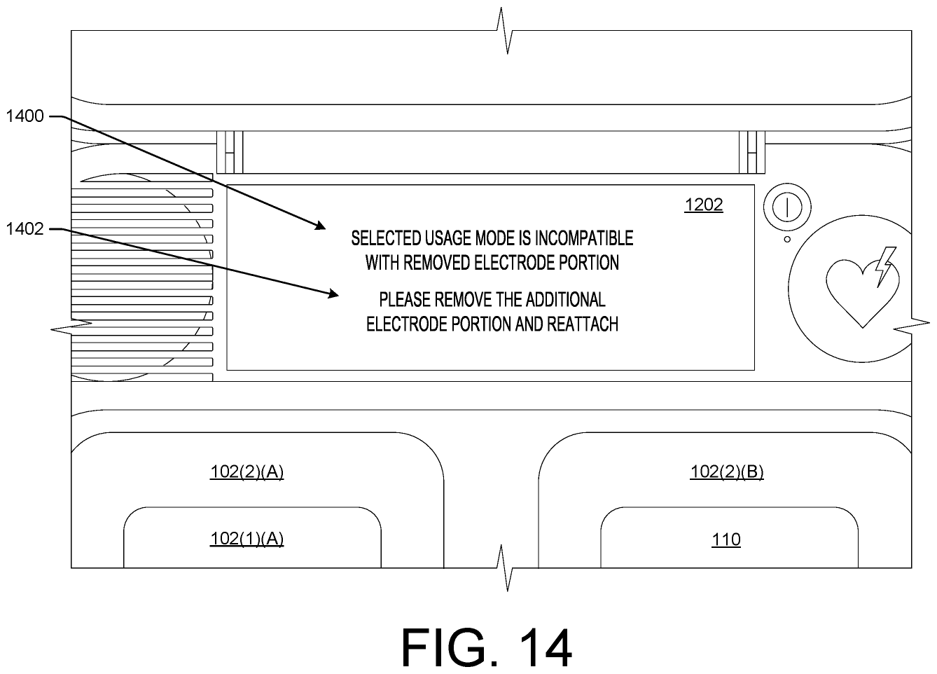

FIG. 14 illustrates a close-up view of the display 1202 of the example external defibrillator 1100 depicted in FIG. 11. The display 1202 in FIG. 14 is outputting an indication 1400 that a user-selected mode is incompatible with the removed electrode portion(s) 102, and a prompt 1402 for the user 400 to re-attach an additional electrode portion(s) 102. Consider an example where the user 400 removes the first electrode portion 102(1)(B) (and possibly the other first electrode portion 102(1)(A)) from the electrode storage tray 1102, and the pediatric mode is automatically selected by the external defibrillator 1100 based on the removed electrode portion(s) 102, and then the user 400 subsequently overrides the device-selected pediatric mode by providing user input to change the usage mode of the external defibrillator 1100 to the adult mode. In this example, the display 1202 (and/or another output device(s) of the external defibrillator 1100) outputs the indication 1400 to indicate to the user 400 that the adult mode selected by the user 400 is incompatible with the first electrode portion 102(1)(B) (and possibly the other first electrode portion 102(1)(A)) that has been removed from the electrode storage tray 1102. If the user 400 inadvertently changed the usage mode to the adult mode, then this indication 1400 provides the user 400 with an opportunity to change the usage mode back to the pediatric mode. Otherwise, if the user 400 intended to change the usage mode to the adult mode in this example, the prompt 1402 provides the user 400 with instructions to resolve the incompatibility. In this example, the incompatibility may be resolved by the user 400 placing the first electrode portion 102(1)(B) back in the electrode storage tray 1102 atop the second electrode portion 102(2)(B) and then removing both electrode portions 102(1)(B) and 102(2)(B) from the electrode storage tray 1102, and this may be repeated with respect to the other electrode assembly if the user 400 removed the first electrode portion 102(1)(A) without removing the second electrode portion 102(2)(A). Alternatively, the incompatibility may be resolved by the user 400 removing the second electrode portion 102(2)(B) from the electrode storage tray 1102 and re-attaching the second electrode portion 102(2)(B) to the first electrode portion 102(1)(B), and this may be repeated with respect to the other electrode assembly if the user 400 removed the first electrode portion 102(1)(A) without removing the second electrode portion 102(2)(A). In an example, the multiple electrode portions 102 of each electrode assembly may be coupled together with an adhesive that retains its adhesive properties even after separation of the electrode portions 102 from one another. This allows the electrode portions 102 to be separated from each other and subsequently re-attached to each other. This gives the user 400 a second chance to remove the appropriate electrode portions 102 from the electrode storage tray 1102 before defibrillating a patient 924.

In some examples, the prompt 1402 may provide a choice for the user 400 to either re-attach an additional electrode portion(s) 102 to the removed electrode portion(s) 102 or to change the usage mode. In some examples, the prompt 1402 provides the user 400 with the choice to change the usage mode from the adult mode back to the pediatric mode. In some examples, a prompt is output that prompts the user 400 to change the usage mode without prompting the user 400 to re-attach the additional electrode portion(s) 102.

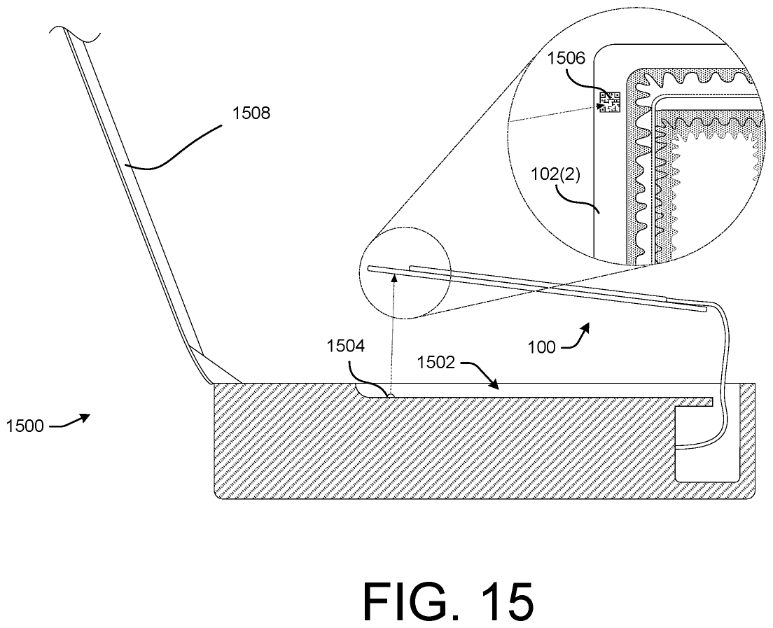

FIG. 15 illustrates a side cross-sectional view of an example external defibrillator 1500, and further depicting an optical sensor 1504 disposed in the electrode storage tray 1502, the optical sensor 1504 being usable for determining whether an electrode portion(s) 102 of the electrode assembly 100 remain(s) disposed in the electrode storage tray 1502. The external defibrillator 1500 may be similar to the external defibrillator 1100. The optical sensor 1504 can be any suitable type of optical sensor including a camera, a photodiode, an ambient light sensor, an infrared (IR) sensor, a barcode scanner, and/or any other type of optical sensor. In the example of FIG. 15, the optical sensor 1504 is configured to detect or sense an optical parameter in the form of a visual indicator 1506 associated with the electrode assembly 100 and/or associated with a particular electrode portion(s) 102 thereof. In this example, the presence of the visual indicator 1506 is indicative of a presence of an associated electrode portion(s) 102 within the electrode storage tray 1502. The visual indicator 1506 can by any suitable type of visual indicator including a Quick Response (QR) code, a barcode, a colored patch, a patterned patch, text (e.g., an alphanumeric code), an image, a symbol, and/or any other type of visual indicator. In some examples, the visual indicator 1506 is printed on a surface (e.g., a bottom surface) of the electrode portion(s) 102. In some examples, a particular electrode portion 102, such as the second electrode portion 102(2) shown in FIG. 15, includes the visual indicator 1506 and other electrode portions 102 of the electrode assembly 100 do not have a visual indicator associated therewith. In other examples, each electrode portion 102 of the electrode assembly 100 includes a visual indicator, such as the visual indicator 1506, which may be used to uniquely identify the associated electrode portion 102. In these examples, the visual indicators 1506 included (e.g., printed) on the electrode portions 102 of a given electrode assembly 100 may be different for each electrode portion 102 (e.g., different QR codes, different color patches, etc.).

Consider an example where the user 400 removes the first electrode portion 102(1) of the electrode assembly 100 from the electrode storage tray 1502, and the second electrode portion 102(2) remains disposed in the electrode storage tray 1502. In this example, the optical sensor 1504 is used to detect the visual indicator 1506 associated with the second electrode portion 102(2). The detection of the visual indicator 1506 is based on image analysis, in some examples. Accordingly, the optical sensor 1504, in some examples, includes an image sensor, and the external defibrillator 1500 includes an image processor (and/or image processing software) to process images acquired by the image sensor. Based on detecting the visual indicator 1506 with the optical sensor 1504, the logic of the external defibrillator 1500 determines that the second electrode portion 102(2) remains disposed in the electrode storage tray 1502. In some examples, the external defibrillator 1500 may also determine that the first electrode portion 102(1) has been removed from the electrode storage tray 1502 based at least in part on detecting the visual indicator 1506 with the optical sensor 1504 and/or based on additional parameter(s) detected or sensed by the optical sensor 1504 and/or by additional optical sensors 1504 disposed in the electrode storage tray 1502. In an example, the optical sensor 1504 may be configured to detect the presence or the absence of more than one electrode portion 102 of the electrode assembly 100, such as by detecting, or failing to detect, a visual indicator associated with a particular electrode portion(s) 102. In this example, the optical sensor 1504 may fail to detect a first visual indicator (not shown in FIG. 15) associated with the first electrode portion 102(1) to determine that the first electrode portion 102(1) has been removed from the electrode storage tray 1502. In some examples, a separate optical sensor may be utilized for this determination. It is to be appreciated that any number of optical sensors, such as the optical sensor 1504, may be used to detect the presence or the absence of any number of electrode portions 102 of an electrode assembly. Accordingly, in an example with a three-portion electrode assembly, a first visual indicator may be associated with a first electrode portion, a second visual indicator, such as the visual indicator 1506, may be associated with the second electrode portion, and a third visual indicator may be associated with the third electrode portion. The external defibrillator 1500 in this example is configured to use the optical sensor 1504 (and/or additional optical sensors) to detect these visual indicators. If, say, the second and third visual indicators are detected by the optical sensor(s) in the electrode storage tray 1502, but the optical sensor(s) fail(s) to detect the first visual indicator, the external defibrillator 1500 may determine that the first electrode portion has been removed from the electrode storage tray 1502 while the second and third electrode portions remain disposed in the electrode storage tray 1502. Although the optical sensor(s) 1504 is depicted in FIG. 15 as being disposed in the electrode storage tray 1502 to optically detect a visual indicator(s) 1506 on a bottom surface of the electrode portion(s) 102, it is to be appreciated that the optical sensor(s) 1504 can be positioned above the electrode portion(s) 102, such as in the lid 1508 of the external defibrillator 1500, to detect a visual indicator(s) 1506 disposed on a top surface of the electrode portion(s) 102. Furthermore, the electrode storage tray 1502 is transparent, in some examples, and the optical sensor(s) 1504 is disposed behind a surface of the electrode storage tray 1502, rather than being disposed within, or extending through, an aperture in the electrode storage tray 1502.

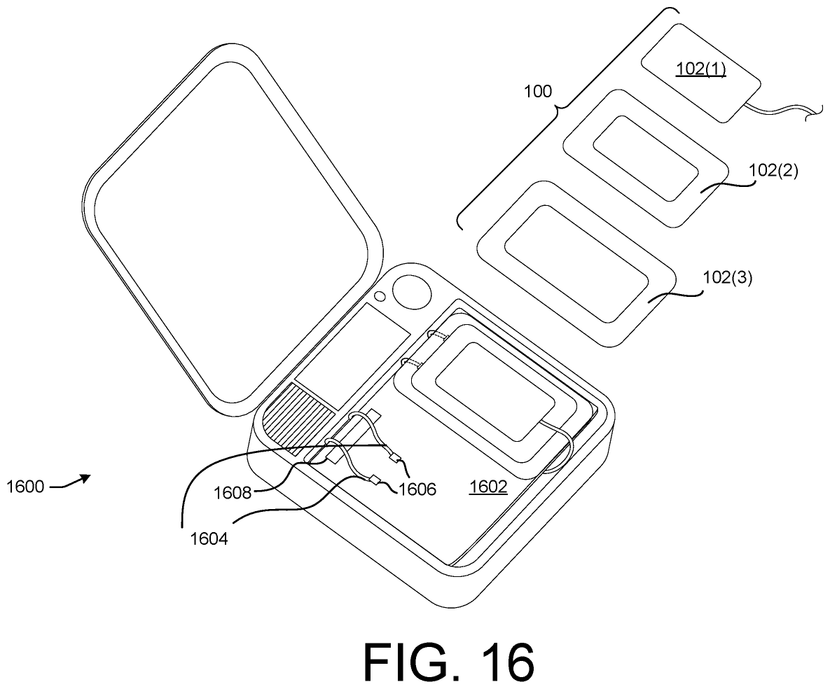

FIG. 16 illustrates a perspective view of an example external defibrillator 1600, and further depicting sense leads 1604 of an electrical circuit disposed in the electrode storage tray 1602, the electrical circuit being usable for determining whether an electrode portion 102 of the electrode assembly 100 remains disposed in the electrode storage tray 1602. The external defibrillator 1600 may be similar to the external defibrillator 1100. In the example of FIG. 16, the electrical circuit, using the sense leads 1604, is configured to detect or sense an electrical parameter(s) indicative of the presence or the absence of an electrode portion(s) in the electrode storage tray 1602. The example of FIG. 16 also depicts a three-portion electrode assembly 100. In this example, a layer of gel disposed on the third electrode portion 102(3) of the electrode assembly 100 forms a conductive bridge between the sense leads 1604 when the electrode assembly is disposed in the electrode storage tray 1602, causing the sense leads (and sensors 1606 attached to the ends of the sense leads 1604) to contact the bottom surface of the largest electrode portion 102, such as the third electrode portion 102(3) in the example of FIG. 16. This conductive bridge between the sense leads 1604 creates a closed circuit when the third electrode portion 102(3) is disposed in the electrode storage tray. Accordingly, the external defibrillator 1600 is configured to determine that the third electrode portion 102(3) remains disposed in the electrode storage tray 1602 based on detecting the closed circuit. Thus, with a two-portion electrode assembly 100, the external defibrillator 1600 may determine, based on the presence of, say, a second electrode portion 102(2) within the electrode storage tray 1602 (the presence detected based on the closed circuit condition), that the first electrode portion 102(1) has been removed from the electrode storage tray 1602. In other words, when the user 400 powers on the external defibrillator 1600 and removes an electrode portion 102 from the electrode storage tray 1602, such as the first electrode portion 102(1), the mere presence of the other electrode portion 102(2) within the electrode storage tray 1602 may be used to determine (e.g., deduce) that the first electrode portion 102(1) has been removed from the electrode storage tray 1602. If, on the other hand, the user 400 removes the entire electrode assembly 100 from the electrode storage tray 1602, the sense leads 1604 (and sensors 1606 attached to the ends of the sense leads 1604) are configured to break away from the bottom surface of the largest electrode portion 102, such as the third electrode portion 102(3) in the example of FIG. 16. This separation of the sense leads 1604 (and sensors 1606) from the third electrode portion 102(3) creates an open circuit. Accordingly, the external defibrillator 1600 is configured to determine that the third electrode portion 102(3) has been removed from the electrode storage tray 1602 based on detecting the open circuit. This approach can be used to determine whether an electrode assembly 100 having any number of electrode portions 102 has been removed from the electrode storage tray 1602.

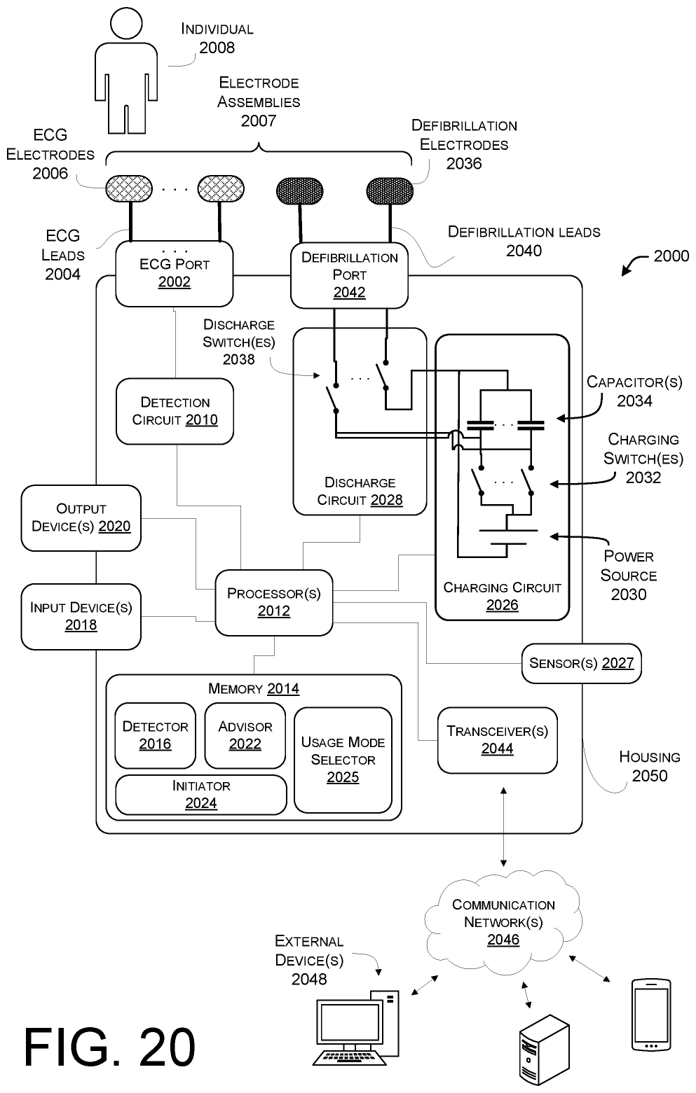

In some examples, the external defibrillator 1600 includes an impedance detection circuit and a conductive element 1608 disposed in the electrode storage tray 1602. In some examples, the impedance detection circuit is internal to the housing of the external defibrillator 1600 (See e.g., the detection circuit 2010 of FIG. 20). In these examples, the impedance detection circuit is configured to detect an impedance value associated with the conductive element 1608. The conductive element 1608 may be a metal bar, or any other suitable electrical conductor with known properties. In this way, the external defibrillator 1600 is configured to determine that the entire electrode assembly 100 is disposed within the electrode storage tray 1602 if the detected impedance value is within a first predetermined range of impedance values. However, if the impedance detection circuit detects an impedance value associated with the conductive element 1608 that falls outside of that first predetermined range, the external defibrillator 1600 may determine, based on this change in impedance, that an electrode portion(s) 102 has/have been removed from the electrode storage tray 1602. The logic of the external defibrillator 1600 may be configured to determine which electrode portion(s) 102 has/have been removed based on the detected impedance value falling within respective value ranges that correspond to each electrode portion 102. For instance, if the impedance detection circuit detects an impedance value associated with the conductive element 1608 that is within a predetermined range of impedance values associated with the removal of the first electrode portion 102(1), the external defibrillator 1600 may determine, based on this detected impedance value, that the first electrode portion 102(1) has been removed from the electrode storage tray 1602, and that the other electrode portion(s) 102 (e.g., the second and third electrode portions 102(2) and 102(3)) remain disposed in the electrode storage tray 1602. In some examples, the sensors 1606 and the sense leads 1604 are printed on a liner (e.g., the liner 104, 704) that is disposed in packaging for the electrode assembly 100, and the sense leads 1604 may be coupled to the impedance detection circuit of the external defibrillator 1600 via the electrical cable 114, 714. In these examples, the electrode storage tray 1602 can be omitted, or the electrode storage tray 1602 may omit the sensors 1606 and the sense leads 1604 because they are disposed in the electrode packaging itself.

In some examples, the impedance detection circuit of the external defibrillator 1600 is configured to monitor a condition of the electrode assembly 100, or the electrode portion(s) 102 thereof, when the external defibrillator 1600 is not in use. For example, during periodic self-checks performed by the external defibrillator 1600, the impedance detection circuit of the external defibrillator 1600 can measure the impedance of the electrode portions 102 and recommend (e.g., via an output device of the external defibrillator 1600) that electrodes be replaced, if, for example, the measured impedance is equal to or greater than a threshold. This may occur because of the gel drying out, or other reasons.

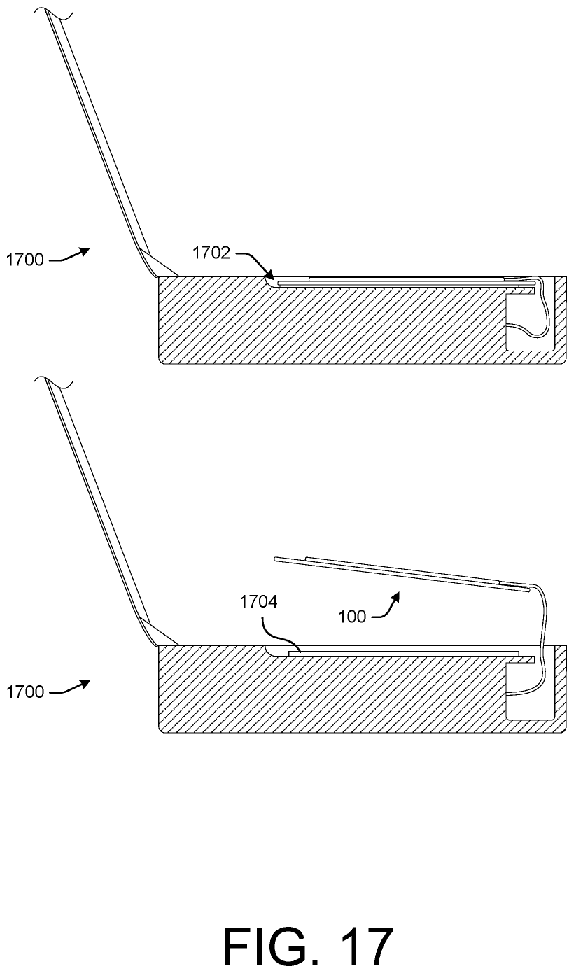

FIG. 17 illustrates a side cross-sectional view of an example external defibrillator 1700, and further depicting a mechanical switch 1704 disposed in the electrode storage tray 1702, the mechanical switch 1704 being usable for determining whether an electrode portion 102 of the electrode assembly 100 remains disposed in the electrode storage tray 1702. The external defibrillator 1700 may be similar to the external defibrillator 1100. The mechanical switch 1704 can be any suitable type of mechanical sensor including a pressure plate, a tact switch, a depressible button, and/or any other type of mechanical sensor. In the example of FIG. 17, the mechanical switch 1704 is configured to detect or sense a mechanical parameter by switching from a first state to a second state in response to removal of an electrode portion(s) 102 from the electrode storage tray 1702. In this example, the mechanical switch 1704 being in the first state is indicative of a presence of an electrode portion(s) 102 within the electrode storage tray 1702. For example, the weight of the second electrode portion 102(2) may be heavy enough to depress the mechanical switch 1704, thereby setting the mechanical switch 1704 to the first state. When the second electrode portion 102(2) is removed from the electrode storage tray 1702, the absence of the second electrode portion 102(2) removes the downward force on the mechanical switch 1704, which causes the mechanical switch to pop-up, thereby switching the mechanical switch 1704 from the first state to the second state in response to the removal of the second electrode portion 102(2) from the electrode storage tray 1702.

It is to be appreciated that any number of mechanical switches, such as the mechanical switch 1704, may be used to detect the presence or the absence of any number of electrode portions 102 of an electrode assembly. Accordingly, in an example with a three-portion electrode assembly, a first mechanical switch may be associated with a first electrode portion, a second mechanical switch may be associated with the second electrode portion, and a third mechanical switch, such as the mechanical switch 1704, may be associated with the third electrode portion. The external defibrillator 1700 in this example is configured to use the mechanical switches to determine which electrode portions 102 remain disposed in the electrode storage tray 1702 and/or which electrode portions 102 have been removed therefrom. If, say, the second and third mechanical switches remain in the first state, but the first mechanical switch has switched to the second state, the external defibrillator 1700 may determine that the first electrode portion has been removed from the electrode storage tray 1702 while the second and third electrode portions remain disposed in the electrode storage tray 1702.

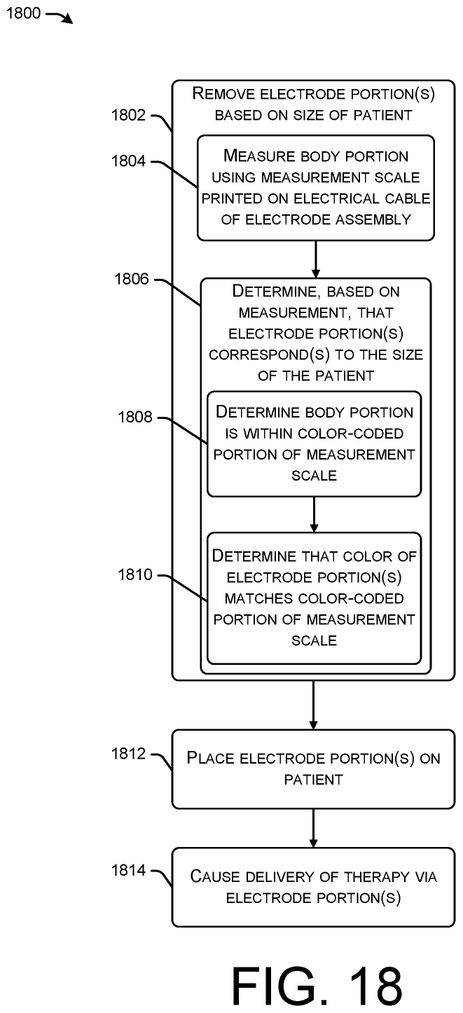

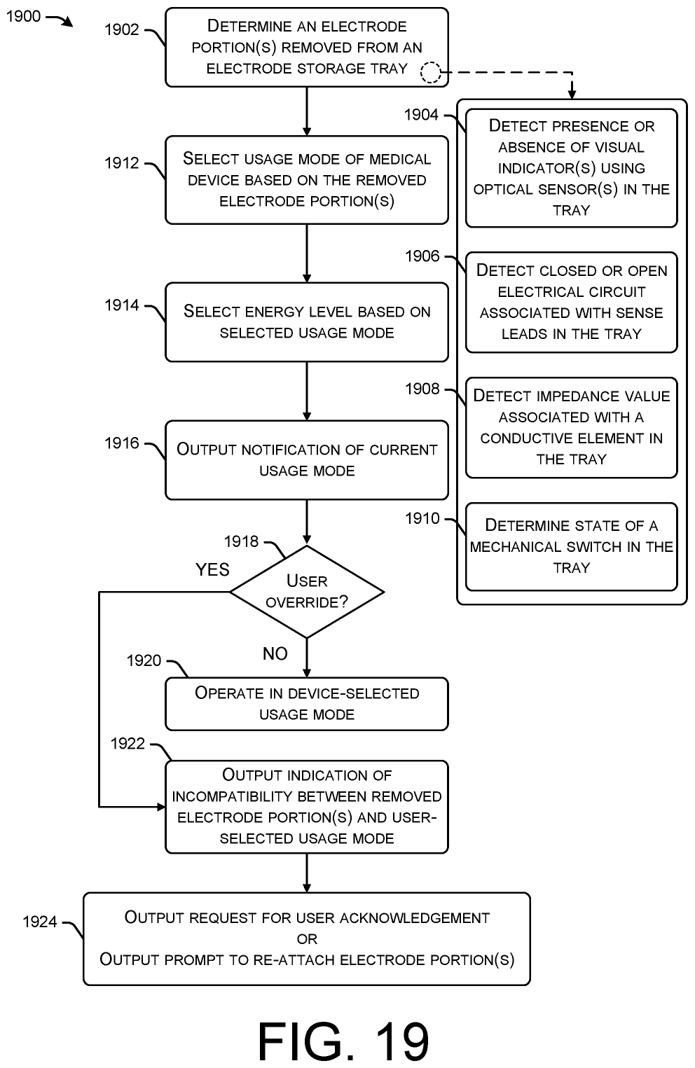

FIGS. 18-19 illustrate example processes related to various implementations of the present disclosure. Although FIGS. 18-19 illustrate separate processes, in various examples, a single entity can perform any combination of the processes, and/or any part of a process. Furthermore, although each of FIGS. 18-19 illustrates steps in a particular order, implementations are not limited to the specific order of operations illustrated in the figures.

FIG. 18 illustrates an example process 1800 for adapting an electrode assembly to a size that is suitable for delivering therapy to a patient. In various implementations, the process 1800 is performed by an entity such as a user 400 of a medical device (e.g., an external defibrillator) that includes an electrode assembly, as described herein.

At 1802, a user 400 removes, based on a size of a patient 924, an electrode portion(s) 102 of an electrode assembly 100 from one or more remaining electrode portions 102 of the electrode assembly 100, as described herein. For example, using a two-portion electrode assembly 100, the user 400 may remove a first electrode portion 102(1) from a second electrode portion 102(2), such as by peeling (e.g., while grasping a pull tab 402) the first electrode portion 102(1) away from the second electrode portion 102(2). As shown in FIG. 18, removing an electrode portion(s) 102 based on the size of the patient 924 may include one or more sub-operations.

At sub-block 1804, for example, the user 400 measures a portion of a body of the patient 924 using a measurement scale printed on an electrical cable 114 of the electrode assembly 100 to obtain a measurement. An example of this is depicted in FIG. 10, where a user 400 uses the electrical cable 1014 to measure a demi-span 1028 of the patient 924.

At sub-block 1806, the user 400 determines, based on the measurement obtained at sub-block 1804, that a particular electrode portion(s) 102 corresponds to the size of the patient 924. For example, the user 400 may determine that the first electrode portion 102(1) corresponds to the size of a pediatric patient 924(2), based on the measurement obtained at sub-block 1804. As shown in FIG. 18, determining that the particular electrode portion(s) 102 corresponds to the size of the patient 924 based on the measurement may include one or more sub-operations.

At sub-block 1808, for example, the user 400 determines that the portion of the body is within a portion 1030 of the measurement scale that is color-coded with a particular color. For example, the user 400 may determine that the portion of the body (e.g., the demi-span 1028) of the patient is within an orange portion 1030 of the measurement scale.

At sub-block 1810, the user 400 determines that a color of a particular electrode portion(s) 102 matches the particular color of the portion 1030 of the measurement scale. For example, the user 400 may identify an orange electrode portion, such as the first electrode portion 102(1) that is colored orange, as a matching electrode portion(s).

With the suitable electrode portion(s) 102 determined and removed from the remaining electrode portion(s) 102, the user 400, at 1812, places the electrode portion(s) 102 on the patient 924. For example, the user 400 may place the removed first electrode portion 102(1) on a pediatric patient 924(2).

At 1814, the user 400 causes delivery of therapy to the patient 924 via a conductive area(s) of the removed electrode portion(s) 102. For example, the user 400 may operate a medical device 1026 (e.g., an external defibrillator) that is coupled to the removed electrode portion(s) 102 via an electrical cable 114 to cause delivery of the therapy (e.g., defibrillation therapy, such as an electrical shock based on an electrical signal received from the medical device 1026) to the patient 924. This may include delivery of therapy via the first conductive area 500(1) of the first electrode portion 102(1), without using the second electrode portion 102(2) (or any other portions 102 for that matter), in an example where the removed electrode portion 102 is the first electrode portion 102(1). Accordingly, the electrode portion(s) 102 in use receive(s) an electrical signal from the medical device 1026 (e.g., an external defibrillator), and the electrode portion(s) 102 delivers an electrical shock via the conductive area(s) of the electrode portion(s) 102 based on the received electrical signal. In some examples, an electrical shock is delivered at an energy level associated with the size and/or age of the patient 924. For instance, the medical device 1026 (e.g., an external defibrillator) and/or the user 400 may select a usage mode and/or an energy level in/at which to operate the medical device 1026, such as a pediatric mode at a relatively low energy level, as compared to an adult mode.

Thus, with the process 1800, a user 400 can conveniently adapt an electrode assembly 100 to a size that is appropriate for a size and/or age of a patient 924 by removing (e.g., peeling away) any number of electrode portions 102, as desired. Alternatively, if the patient 924 is greater than a threshold size and/or age, the entire electrode assembly 100 may be used to deliver therapy to the patient 924 without removal of an electrode portion 102, as described herein.

FIG. 19 illustrates an example process 1900 for selecting a usage mode of a medical device (e.g., an external defibrillator) based on the removal of one or more electrode portions of an electrode assembly from an electrode storage tray of the medical device. In various implementations, the process 1900 is performed by an entity such as a medical device (e.g., one or more processors thereof) that includes an electrode assembly, as described herein.

At 1902, a medical device 1026 (e.g., an external defibrillator 1100) determines that an electrode portion(s) 102 of an electrode assembly 100 has/have been removed from an electrode storage tray 1102 of the medical device 1026. The determination at 1902 may include determining that one or more remaining electrode portions 102 of the electrode assembly 100 remain disposed in the electrode storage tray 1102. In an example with a two-portion electrode assembly 100, the medical device 1026 may determine that the first electrode portion 102(1) of the electrode assembly 100 has been removed from the electrode storage tray 1102 and that the second electrode portion 102(2) of the electrode assembly 100 remains disposed in the electrode storage tray 1102. As shown in FIG. 19, determining that an electrode portion(s) 102 has/have been removed from the electrode storage tray 1102 may include one or more sub-operations.

At sub-block 1904, for example, an optical sensor(s) 1504 disposed within the electrode storage tray detect(s), after the electrode portion(s) 102 has/have been removed from the electrode storage tray, a visual indicator(s) 1506 associated with the remaining electrode portion(s) 102 that remain(s) disposed in the electrode storage tray. Continuing with the example of a two-portion electrode assembly 100, after the first electrode portion 102(1) has been removed from the electrode storage tray, the optical sensor(s) 1504 may detect a visual indicator 1506 associated with the second electrode portion 102(2) that remains in the electrode storage tray. In some examples, there are multiple optical sensors, such as the optical sensor 1504, disposed within the electrode storage tray of the medical device 1026, each optical sensor corresponding to an electrode portion 102 of the electrode assembly 100. In these examples, the operations at sub-block 1904 may include one or more optical sensors 1504 failing to detect a visual indicator(s) 1506 of a corresponding electrode portion(s) 102, and one or more other optical sensors 1504 detecting a visual indicator(s) 1506 of another corresponding electrode portion(s) 102. With an example of a three-portion electrode assembly 100, an optical sensor(s) 1504 may fail to detect a first visual indicator 1506 associated with the first electrode portion 102(1), the same or a different optical sensor(s) 1504 may detect a second visual indicator 1506 associated with the second electrode portion 102(2), and the same or a different optical sensor(s) 1504 may detect a third visual indicator 1506 associated with the third electrode portion 102(3). In this example, the failure to optically detect the first visual indicator 1506 and the optical detection of the second and third visual indicators 1506 informs the medical device 1026 that the first electrode portion 102(1) has been removed from the electrode storage tray, and that the second and third electrode portions 102(2) and 102(3) remain disposed in the electrode storage tray.

At sub-block 1906, as another example, the medical device 1026 detects, after the electrode portion(s) 102 has/have been removed from the electrode storage tray, a closed circuit created by a layer of gel—disposed on the remaining electrode portion(s) 102 that remain(s) disposed in the electrode storage tray—forming a conductive bridge between sense leads 1604 of an electrical circuit disposed in the electrode storage tray. With the example of a two-portion electrode assembly 100, after the first electrode portion 102(1) has been removed from the electrode storage tray, the medical device 1026 may detect a closed circuit created by a layer of gel disposed on the second electrode portion 102(2) forming a conductive bridge between sense leads 1604 of an electrical circuit disposed in the electrode storage tray. This closed circuit condition informs the medical device 1026 that the second electrode portion 102(2) remains disposed in the electrode storage tray after removal of the first electrode portion 102(1) therefrom.

At sub-block 1908, as yet another example, the medical device 1026 includes an impedance detection circuit, and the bottom surface of the largest electrode portion 102 is in contact with a conductive element 1608 (e.g., a metal bar) while the largest electrode portion 102 is disposed in the electrode storage tray. With an example of a three-portion electrode assembly 100, the impedance detection circuit of the medical device 1026 may detect an impedance value associated with the conductive element 1608 disposed in the electrode storage tray, and the medical device 1026 may determine that the impedance value is within a predetermined range of impedance values, the predetermined range of impedance values indicating that the first electrode portion 102(1) has been removed from the electrode storage tray and that the second and third electrode portions 102(2) and 102(3) remain disposed in the electrode storage tray. In this manner, multiple different predetermined ranges of impedance values can be defined and utilized to determine which electrode portion(s) 102 has/have been removed from the electrode storage tray based on a detected impedance value associated with the conductive element 1608.

At sub-block 1910, the medical device 1026 determines, after the electrode portion(s) 102 has/have been removed from the electrode storage tray, that a mechanical switch(es) 1704 disposed in the electrode storage tray remains in a first state of two states. This/these mechanical switch(es) 1704 may be associated with the remaining electrode portion(s) 102 that remain(s) disposed in the electrode storage tray, and may be configured to switch (or transition) between two states: a first state and a second state. In some examples, the first state is a depressed state where the mechanical switch 1704 is in a first (e.g., depressed) position, and the second state is an extended state where the mechanical switch 1704 is in a second (e.g., extended) position. An example of the mechanical switch 1704 in the second (e.g., extended) position is depicted in FIG. 17. Accordingly, with an example of a two-portion electrode assembly 100, if the medical device 1026 determines, after the first electrode portion 102(1) has been removed from the electrode storage tray, that a mechanical switch 1704 disposed in the electrode storage tray and associated with the second electrode portion 102(2) remains in a first (e.g., depressed) state of two states, the medical device 1026 can determine, based on the first state of the mechanical switch 1704, that the second electrode portion 102(2) remains disposed in the electrode storage tray and that the first electrode portion 102(1) has been removed therefrom. In some examples, there are multiple mechanical switches, such as the mechanical switch 1704, disposed within the electrode storage tray of the medical device 1026, each mechanical switch corresponding to an electrode portion 102 of the electrode assembly 100. In these examples, the operations at sub-block 1910 may include one or more mechanical switches 1704 remaining in the first state of the two states, and one or more other mechanical switches 1704 switching to the second state of the two states. With an example of a three-portion electrode assembly 100, a first mechanical switch 1704 disposed in the electrode storage tray and associated with the first electrode portion 102(1) may have switched to the second state of the two states, and the second and third mechanical switches 1704 disposed in the electrode storage tray and associated with the second and third electrode portions 102(2) and 102(3), respectively, may remain in the first state. In this example, the second and third mechanical switches 1704 remaining in the first state, and the first mechanical switch 1704 having switched to the first state informs the medical device 1026 that the first electrode portion 102(1) has been removed from the electrode storage tray, and that the second and third electrode portions 102(2) and 102(3) remain disposed in the electrode storage tray.

At 1912, the medical device 1026 selects, based on the electrode portion(s) 102 having been removed from the electrode storage tray and the remaining electrode portion(s) remaining disposed in the electrode storage tray, a usage mode of a plurality of usage modes in which the medical device 1026 is to operate. That is the medical device 1026 may select (e.g., automatically, without user intervention) a particular usage mode depending on which electrode portion(s) 102 have been removed. With an example of a two-portion electrode assembly 100, if the first electrode portion 102(1) is removed from the electrode storage tray and the second electrode portion 102(2) remains disposed in the electrode storage tray, the medical device 1026 may select a pediatric mode, among possible usage modes including the pediatric mode and an adult mode. In other examples, the electrode assembly 100 may include more than two electrode portions 102 (e.g., three electrode portions, four electrode portions, etc.), and the medical device 1026 may be configured to operate in more than two usage modes, such as an infant mode, a pediatric mode, an adult mode, etc. In the three-portion electrode assembly 100 example, if the first electrode portion 102(1) is removed from the electrode storage tray and the second and third electrode portions 102(2) and 102(3) remain disposed in the electrode storage tray, the medical device 1026 may select an infant mode, among possible usage modes including the infant mode, a pediatric mode, and an adult mode

At 1914, the medical device 1026 selects, based on the usage mode selected at block 1912, an energy level associated with an electrical signal to be sent to the first electrode portion 102(1) of the electrode assembly 100. For example, the medical device 1026 may be a defibrillator 1100, and the defibrillator 1100 is configured to select (automatically, without user intervention) a particular energy level at which to deliver electrical signals to the first electrode portion 102(1), depending on the usage mode selected at block 1912. For instance, the defibrillator 1100 may deliver electrical signals to the first electrode portion 102(1) at a first energy level while in pediatric mode, and at a second energy level higher than the first energy level while in adult mode. In general, the energy level may be proportional to the size and/or age of the patient 924 such that relatively lower energy levels are appropriate and/or safe for smaller and/or younger patients 924 while relatively higher energy levels are appropriate and/or safe for larger and/or older patients 924. Furthermore, the electrical signal is delivered at the selected energy level to the first electrode portion 102(1), and due to the electrical conductivity between the first electrode portion 102(1) and one or more other electrode portions 102 (if other electrode portions 102 are being used together with the first electrode portion 102(1)), the conductive areas of each electrode portion 102 may deliver therapy (e.g., defibrillation therapy, such as an electrical shock) to the patient 924. The electrical conductivity between electrode portions 102 may be provided by a layer of gel 600 and/or by one or more conductive elements 716 that act(s) as an electrical bridge between the respective conductive areas of the electrode portions 102 (if more than one electrode portion 102 is being used to deliver therapy to the patient 924).

At 1916, the medical device 1026 outputs, via an output device of the medical device 1026, a notification that the medical device 1026 is in the usage mode selected at block 1912. The output device may be a display(s) 1200, a speaker(s), one or more light indicators (e.g., colored LEDs), a haptic actuator (e.g., vibratory mechanism), and/or any other suitable output device. Likewise, the notification may be output visually, audibly, and/or vibrationally.

At 1918, the medical device 1026 determines whether a user 400 has provided user input to override the device-selected usage mode. If no such user input is received by the medical device 1026, the process 1900 follows the NO route from block 1918 to block 1920 where the medical device 1026 operates in the device-selected usage mode, such as to deliver therapy (e.g., defibrillation therapy) to the patient 924. In an example, the device-selected usage mode may be the pediatric mode, if, say, the user 400 removed the first electrode portion 102(1) of a two-portion electrode assembly 100. If, at 1918, the medical device 1026 receives, via an input device (e.g., a touch-sensitive display 1200, a button, and/or a microphone(s), etc.) of the medical device 1026, user input to select a user-selected usage mode of the plurality of usage modes in which the medical device 1026 is to operate, the process 1900 follows the YES route from block 1918 to block 1922.

At 1922, the medical device 1026 outputs, via an output device (e.g., a display 1200, a speaker(s), a light indicator(s), and/or a haptic mechanism, etc.) of the medical device 1026, an indication that the user-selected usage mode selected by a user 400 of the medical device 1026 is incompatible with the electrode portion(s) 102 that has/have been removed from the electrode storage tray. In an example, the user 400 may have removed the first electrode portion 102(1) of a two-portion electrode assembly 100, the medical device 1026 may have automatically selected the pediatric mode, and the user 400 may have provided user input to override the device-selected usage mode by selecting the adult mode. In this example, the indication output by the medical device 1026 at 1922 may indicate that the adult mode selected by the user 400 is incompatible with the first electrode portion 102(1) that has been removed from the electrode storage tray.

At 1924, the medical device 1026 outputs, via an output device (e.g., a display 1200, a speaker(s), a light indicator(s), and/or a haptic mechanism, etc.) of the medical device 1026, a request for the user 400 to acknowledge that the user-selected usage mode is incompatible with the removed electrode portion(s) 102. An example of this request 1302 is illustrated in FIG. 13. Additionally, or alternatively, the medical device 1026 outputs a prompt for the user 400 to remove the other electrode portion(s) 102 from the electrode storage tray and to re-attach that/those electrode portion(s) 102 to the removed electrode portion(s) 102. An example of this prompt 1402 is illustrated in FIG. 14.

FIG. 20 illustrates an example of an external defibrillator 2000 having an electrode assembly described herein, and configured to perform various functions described herein. For example, the external defibrillator 2000 is an example of the medical device 1026 described elsewhere herein, and, in some examples, the external defibrillator 2000 represents any of the external defibrillators 1100, 1500, 1600, 1700 described elsewhere herein.

The external defibrillator 2000 includes an ECG port 2002 connected to multiple ECG leads 2004. In some cases, the ECG leads 2004 are removable from the ECG port 2002. For instance, the ECG leads 2004 are plugged into the ECG port 2002. The ECG leads 2004 are connected to ECG electrodes 2006, respectively. In various implementations, the ECG electrodes 2006 are disposed on different locations on an individual 2008 (e.g., a patient 924). A detection circuit 2010 is configured to detect relative voltages between the ECG electrodes 2006. These voltages are indicative of the electrical activity of the heart of the individual 2008.

In various implementations, the ECG electrodes 2006 are in contact with the different locations on the skin of the individual 2008. In some examples, a first one of the ECG electrodes 2006 may represent one or more electrode assemblies 2007, which may represent any of the electrode assemblies 100, 700, 800, 900, 1000, as described elsewhere herein. In some examples, a first one of the ECG electrodes 2006 is placed on the skin between the heart and right arm of the individual 2008, a second one of the ECG electrodes 2006 is placed on the skin between the heart and left arm of the individual 2008, and a third one of the ECG electrodes 2006 is placed on the skin between the heart and a leg (either the left leg or the right leg) of the individual 2008. In these examples, the detection circuit 2010 is configured to measure the relative voltages between the first, second, and third ECG electrodes 2006. Respective pairings of the ECG electrodes 2006 are referred to as “leads,” and the voltages between the pairs of ECG electrodes 2006 are known as “lead voltages.” In some examples, more than three ECG electrodes 2006 are included, such that 5-lead or 12-lead ECG signals are detected by the detection circuit 2010.

The detection circuit 2010 includes at least one analog circuit, at least one digital circuit, or a combination thereof. The detection circuit 2010 receives the analog electrical signals from the ECG electrodes 2006, via the ECG port 2002 and the ECG leads 2004. In some cases, the detection circuit 2010 includes one or more analog filters configured to filter noise and/or artifact from the electrical signals. The detection circuit 2010 includes an analog-to-digital converter (ADC) in various examples. The detection circuit 2010 generates a digital signal indicative of the analog electrical signals from the ECG electrodes 2006. This digital signal can be referred to as an “ECG signal” or an “ECG.”

In some cases, the detection circuit 2010 further detects an electrical impedance between at least one pair of the ECG electrodes 2006. For example, the detection circuit 2010 includes, or otherwise controls, a power source that applies a known voltage across a pair of the ECG electrodes 2006 and detects a resultant current between the pair of the ECG electrodes 2006. The impedance is generated based on the applied voltage and the resultant current. In various cases, the impedance corresponds to respiration of the individual 2008, chest compressions performed on the individual 2008, and other physiological states of the individual 2008. In various examples, the detection circuit 2010 includes one or more analog filters configured to filter noise and/or artifact from the resultant current. The detection circuit 2010 generates a digital signal indicative of the impedance using an ADC. This digital signal can be referred to as an “impedance signal” or an “impedance.” In some examples, the detection circuit 2010 includes or represents an impedance detection circuit described elsewhere herein that is configured to detect an impedance value associated with a conductive element 1608 disposed in the electrode storage tray of the external defibrillator 2000 for purposes of determining which electrode portion(s) 102 have been removed from the electrode storage tray.

The detection circuit 2010 provides the ECG signal and/or the impedance signal to one or more processors 2012 in the external defibrillator 2000. In some implementations, the processor(s) 2012 includes a central processing unit (CPU), a graphics processing unit (GPU), both CPU and GPU, or other processing unit or component known in the art.

The processor(s) 2012 is operably connected to memory 2014. In various implementations, the memory 2014 includes volatile (such as random access memory (RAM)), non-volatile (such as read only memory (ROM), flash memory, etc.) or some combination of the two. The memory 2014, in some examples, further includes “working” memory, such as RAM, where applications are loaded for execution on the defibrillator 2000. In general, the memory 2014 stores instructions that, when executed by the processor(s) 2012, causes the processor(s) 2012 to perform various operations. In various examples, the memory 2014 stores methods, threads, processes, applications, objects, modules, any other sort of executable instruction, or a combination thereof. In some cases, the memory 2014 stores files, databases, or a combination thereof. In some examples, the memory 2014 includes, but is not limited to, RAM, ROM, electrically erasable programmable read-only memory (EEPROM), flash memory, or any other memory technology. In some examples, the memory 2014 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 2012 and/or the external defibrillator 2000. In some cases, the memory20814 at least temporarily stores the ECG signal and/or the impedance signal.

In various examples, the memory 2014 includes a detector 2016, which causes the processor(s) 2012 to determine, based on the ECG signal and/or the impedance signal, whether the individual 2008 is exhibiting a particular heart rhythm. For instance, the processor(s) 2012 determines whether the individual 2008 is experiencing a shockable rhythm that is treatable by defibrillation. Examples of shockable rhythms include ventricular fibrillation (VF) and pulseless ventricular tachycardia (V-Tach). In some examples, the processor(s) 2012 determines whether any of a variety of different rhythms (e.g., asystole, sinus rhythm, atrial fibrillation (AF), etc.) are present in the ECG signal.

The processor(s) 2012 is operably connected to one or more input devices 2018 and one or more output devices 2020. Collectively, the input device(s) 2018 and the output device(s) 2020 function as an interface between a user and the defibrillator 2000. The input device(s) 2018 is configured to receive an input from a user and includes at least one of a keypad, a cursor control, a touch-sensitive display, a voice input device (e.g., a speaker), a haptic feedback device, or any combination thereof. The output device(s) 2020 includes at least one of a display (e.g., the display 1200), a speaker, a haptic output device, a printer, or any combination thereof. In various examples, the processor(s) 2012 causes a display to visually output a waveform of the ECG signal and/or the impedance signal. In some implementations, the input device(s) 2018 includes one or more touch sensors, the output device(s) 2020 includes a display screen, and the touch sensor(s) are integrated with the display screen. Thus, in some cases, the external defibrillator 2000 includes a touchscreen configured to receive user input signal(s) and visually output physiological parameters, such as the ECG signal and/or the impedance signal.

In some examples, the memory 2014 includes an advisor 2022, which, when executed by the processor(s) 2012, causes the processor(s) 2012 to generate advice and/or control the output device(s) 2020 to output the advice to a user (e.g., a rescuer). In some examples, the processor(s) 2012 provides, or causes the output device(s) 2020 to provide, an instruction to perform CPR on the individual 2008. In some cases, the processor(s) 2012 evaluates, based on the ECG signal, the impedance signal, or other physiological parameters, CPR being performed on the individual 2008 and causes the output device(s) 2020 to provide feedback about the CPR in the instruction. According to some examples, the processor(s) 2012, upon identifying that a shockable rhythm is present in the ECG signal, causes the output device(s) 2020 to output an instruction and/or recommendation to administer a defibrillation shock to the individual 2008.

The memory 2014 also includes an initiator 2024 which, when executed by the processor(s) 2012, causes the processor(s) 2012 to control other elements of the external defibrillator 2000 in order to administer a defibrillation shock to the individual 2008. In some examples, the processor(s) 2012 executing the initiator 2024 selectively causes the administration of the defibrillation shock based on determining that the individual 2008 is exhibiting the shockable rhythm and/or based on an input from a user (received, e.g., by the input device(s) 2018). In some cases, the processor(s) 2012 causes the defibrillation shock to be output at a particular time, which is determined by the processor(s) 2012 based on the ECG signal and/or the impedance signal.

The memory 2014 also includes a usage mode selector 2025 for automatically selecting a usage mode of the external defibrillator 2000 based at least in part on determining which electrode portion(s) 102 has/have been removed from the electrode storage tray of the external defibrillator 2000. The usage mode selector 2025 may be configured to utilize the detection circuit 2010 (e.g., impedance detection circuit) and/or one or more sensors 2027 (e.g., optical sensor(s) 1504, sense leads 1604, and/or mechanical switch(es) 1704, etc.) of the external defibrillator 2000 to determine which electrode portion(s) 102 has/have been removed from the electrode storage tray, as described elsewhere herein.

The processor(s) 2012 is operably connected to a charging circuit 2026 and a discharge circuit 2028. In various implementations, the charging circuit 2026 includes a power source 2030, one or more charging switches 2032, and one or more capacitors 2034. The power source 2030 includes, for instance, a battery. The processor(s) 2012 initiates a defibrillation shock by causing the power source 2030 to charge at least one capacitor among the capacitor(s) 2034. For example, the processor(s) 2012 activates at least one of the charging switch(es) 2032 in the charging circuit 2026 to complete a first circuit connecting the power source 2030 and the capacitor 2034 to be charged. Then, the processor(s) 2012 causes the discharge circuit 2028 to discharge energy stored in the charged capacitor 2034 across a pair of defibrillation electrodes 2036, which are in contact with the individual 2008. In some examples, a first one of the defibrillation electrodes 2036 may represent one or more electrode assemblies 2007, which may represent any of the electrode assemblies 100, 700, 800, 900, 1000, as described elsewhere herein. For example, the processor(s) 2012 deactivates the charging switch(es) 2032 completing the first circuit between the capacitor(s) 2034 and the power source 2030, and activates one or more discharge switches 2038 completing a second circuit connecting the charged capacitor 2034 and at least a portion of the individual 2008 disposed between defibrillation electrodes 2036.

The energy is discharged from the defibrillation electrodes 2036 in the form of a defibrillation shock. For example, the defibrillation electrodes 2036 are connected to the skin of the individual 2008 and located at positions on different sides of the heart of the individual 2008, such that the defibrillation shock is applied across the heart of the individual 2008. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 joules (J) or greater with a duration of about 0.015 seconds. In some cases, the defibrillation shock has a multiphasic (e.g., biphasic) waveform. The discharge switch(es) 2038 are controlled by the processor(s) 2012, for example. In various implementations, the defibrillation electrodes 2036 are connected to defibrillation leads 2040. The defibrillation leads 2040 are connected to a defibrillation port 2042, in implementations. According to various examples, the defibrillation leads 2040 are removable from the defibrillation port 2042. For example, the defibrillation leads 2040 are plugged into the defibrillation port 2042. In some examples, the defibrillation leads 2040 and/or the ECG leads 2004 represent any of the electrical cables 114, 714, 814, 914, 1014 described elsewhere herein.

In various implementations, the processor(s) 2012 is operably connected to one or more transceivers 2044 that transmit and/or receive data over one or more communication networks 2046. For example, the transceiver(s) 2044 includes a network interface card (NIC), a network adapter, a local area network (LAN) adapter, or a physical, virtual, or logical address to connect to the various external devices and/or systems. In various examples, the transceiver(s) 2044 includes any sort of wireless transceivers capable of engaging in wireless communication (e.g., radio frequency (RF) communication). For example, the communication network(s) 2046 includes one or more wireless networks that include a 3^{rd} Generation Partnership Project (3GPP) network, such as a Long Term Evolution (LTE) radio access network (RAN) (e.g., over one or more LE bands), a New Radio (NR) RAN (e.g., over one or more NR bands), or a combination thereof. In some cases, the transceiver(s) 2044 includes other wireless modems, such as a modem for engaging in WI-FI®, WIGIG®, WIMAX®, BLUETOOTH®, or infrared communication over the communication network(s) 2046.

The defibrillator 2000 is configured to transmit and/or receive data (e.g., ECG data, impedance data, data indicative of one or more detected heart rhythms of the individual 2008, data indicative of one or more defibrillation shocks administered to the individual 2008, etc.) with one or more external devices 2048 via the communication network(s) 2046. The external devices 2048 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 2046. In some examples, the external device(s)20848 is located remotely from the defibrillator 2000, such as at a remote clinical environment (e.g., a hospital). According to various implementations, the processor(s) 2012 causes the transceiver(s) 2044 to transmit data to the external device(s) 2048. In some cases, the transceiver(s) 2044 receives data from the external device(s) 2048 and the transceiver(s) 2044 provide the received data to the processor(s) 2012 for further analysis.

In various implementations, the external defibrillator 2000 also includes a housing 2050 that at least partially encloses other elements of the external defibrillator 2000. For example, the housing 2050 encloses the detection circuit 2010, the processor(s) 2012, the memory 2014, the charging circuit 2026, the transceiver(s) 2044, or any combination thereof. In some cases, the input device(s) 2018, output device(s) 2020, and/or the sensor(s) 2027 extend from an interior space at least partially surrounded by the housing 2050 through a wall of the housing 2050. In various examples, the housing 2050 acts as a barrier to moisture, electrical interference, and/or dust, thereby protecting various components in the external defibrillator 2000 from damage.

In some implementations, the external defibrillator 2000 is an automated external defibrillator (AED) operated by an untrained user (e.g., a bystander, layperson, etc.) and can be operated in an automatic mode. In automatic mode, the processor(s) 2012 automatically identifies a rhythm in the ECG signal, makes a decision whether to administer a defibrillation shock, charges the capacitor(s) 2034, discharges the capacitor(s) 2034, or any combination thereof. In some cases, the processor(s) 2012 controls the output device(s) 2020 to output (e.g., display) a simplified user interface to the untrained user. For example, the processor(s) 2012 refrains from causing the output device(s) 2020 to display a waveform of the ECG signal and/or the impedance signal to the untrained user, in order to simplify operation of the external defibrillator 2000.

In some examples, the external defibrillator 2000 is a monitor-defibrillator utilized by a trained user (e.g., a clinician, an emergency responder, etc.) and can be operated in a manual mode or the automatic mode. When the external defibrillator 2000 operates in manual mode, the processor(s) 2012 cause the output device(s) 2020 to display a variety of information that is relevant to the trained user, such as waveforms indicating the ECG data and/or impedance data, notifications about detected heart rhythms, and the like.

### Example Clauses

1. An electrode assembly for an external defibrillator, the electrode assembly comprising: a first electrode portion; and a second electrode portion disposed on the first electrode portion at an edge of the first electrode portion, wherein the second electrode portion has a cutout, and wherein the first electrode portion spans the cutout.
2. The electrode assembly of clause 1, wherein the cutout is centered within the second electrode portion.
3. The electrode assembly of clause 1 or 2, wherein the second electrode portion extends beyond the edge of the first electrode portion.
4. The electrode assembly of any one of clauses 1 to 3, wherein the edge of the first electrode portion is an outer edge of the first electrode portion, and wherein the outer edge of the first electrode portion is horizontally offset from an inner edge of the second electrode portion such that the first electrode portion overlaps part of the second electrode portion, the inner edge of the second electrode portion defining the cutout of the second electrode portion.
5. The electrode assembly of any one of clauses 1 to 4, wherein the second electrode portion is disposed on the first electrode portion at a periphery of the first electrode portion, the periphery of the first electrode portion including the edge of the first electrode portion.
6. The electrode assembly of any one of clauses 1 to 5, wherein the second electrode portion is coupled to the first electrode portion with an adhesive.
7. The electrode assembly of any one of clauses 1 to 6, wherein the first electrode portion is removable from the second electrode portion.
8. The electrode assembly of clause 7, wherein the first electrode portion is configured to be used without the second electrode portion to deliver defibrillation therapy to a pediatric patient.
9. The electrode assembly of clause 7 or 8, wherein the first electrode portion is removable from the second electrode portion by peeling the first electrode portion away from the second electrode portion.
10. The electrode assembly of any one of clauses 1 to 9, wherein the first electrode portion comprises a pull tab.
11. The electrode assembly of clause 10, wherein the pull tab is configured to be grasped by a user to peel the first electrode portion away from the second electrode portion.
12. The electrode assembly of any one of clauses 1 to 11, wherein: the first electrode portion comprises a first conductive area; the second electrode portion comprises a second conductive area; the electrode assembly further comprises a layer of gel disposed on the first conductive area and the second conductive area; and the layer of gel is perforated.
13. The electrode assembly of any one of clauses 1 to 12, wherein: the first electrode portion comprises a first conductive area; the second electrode portion comprises a second conductive area; and the first electrode portion and the second electrode portion are configured to be used together to deliver defibrillation therapy to an adult patient.
14. The electrode assembly of clause 13, further comprising: an electrical cable coupled to the first electrode portion at a first end of the electrical cable, wherein the electrical cable is configured to be coupled to the external defibrillator at a second end of the electrical cable; and a layer of gel disposed on the first conductive area and the second conductive area, wherein the layer of gel provides electrical conductivity between the first electrode portion and the second electrode portion.
15. The electrode assembly of clause 13, further comprising: an electrical cable coupled to the first electrode portion at a first end of the electrical cable, wherein the electrical cable is configured to be coupled to the external defibrillator at a second end of the electrical cable; and a conductive element coupling the first conductive area to the second conductive area to provide electrical conductivity between the first electrode portion and the second electrode portion.
16. The electrode assembly of clause 13, further comprising: a first layer of gel disposed on the first conductive area; a conductive film disposed on the first layer of gel; and a second layer of gel disposed on the second conductive area and the conductive film.
17. The electrode assembly of any one of clauses 1 to 16, wherein the cutout is a first cutout, and the edge is a first edge, the electrode assembly further comprising: a third electrode portion disposed on the second electrode portion at a second edge of the second electrode portion, the third electrode portion having a second cutout, and the first electrode portion and the second electrode portion spanning the second cutout.
18. The electrode assembly of clause 17, wherein: the first electrode portion comprises a first conductive area; the second electrode portion comprises a second conductive area; the third electrode portion comprises a third conductive area; and the first electrode portion, the second electrode portion, and the third electrode portion are configured to be used together to deliver defibrillation therapy to an adult patient.
19. The electrode assembly of clause 17, wherein: the first electrode portion comprises a first conductive area and is removable from the second electrode portion and from the third electrode portion and is configured to be used without the second electrode portion and without the third electrode portion to deliver defibrillation therapy to an infant patient; and the second electrode portion comprises a second conductive area and is removable from the third electrode portion and is configured to be used with the first electrode portion and without the third electrode portion to deliver defibrillation therapy to a pediatric patient.
20. The electrode assembly of clause 17, 18, or 19, further comprising: a fourth electrode portion disposed on the third electrode portion at a third edge of the third electrode portion, the fourth electrode portion having a third cutout, and the first electrode portion, the second electrode portion, and the third electrode portion spanning the third cutout.
21. The electrode assembly of clause 20, wherein: the first electrode portion comprises a first conductive area; the second electrode portion comprises a second conductive area; the third electrode portion comprises a third conductive area; the fourth electrode portion comprises a fourth conductive area; and the first electrode portion, the second electrode portion, the third electrode portion, and the fourth electrode portion are configured to be used together to deliver defibrillation therapy to an adult patient who is greater than a threshold size.
22. The electrode assembly of clause 20, wherein: the first electrode portion comprises a first conductive area and is removable from the second electrode portion, from the third electrode portion, and from the fourth electrode portion and is configured to be used without the second electrode portion, without the third electrode portion, and without the fourth electrode portion to deliver defibrillation therapy to an infant patient; the second electrode portion comprises a second conductive area and is removable from the third electrode portion and from the fourth electrode portion and is configured to be used with the first electrode portion, without the third electrode portion, and without the fourth electrode portion to deliver defibrillation therapy to a pediatric patient; and the third electrode portion comprises a third conductive area and is removable from the fourth electrode portion and is configured to be used with the first electrode portion, with the second electrode portion, and without the fourth electrode portion to deliver defibrillation therapy to an adult patient who is less than a threshold size.
23. An electrode assembly comprising: a first electrode portion; and a second electrode portion disposed on the first electrode portion at an edge of the first electrode portion, the second electrode portion having a cutout, and the first electrode portion spanning the cutout.
24. The electrode assembly of clause 23, wherein the cutout is centered within the second electrode portion.
25. The electrode assembly of clause 23 or 24, wherein the second electrode portion extends beyond the edge of the first electrode portion.
26. The electrode assembly of any one of clauses 23 to 25, wherein the edge of the first electrode portion is an outer edge of the first electrode portion, and wherein the outer edge of the first electrode portion is horizontally offset from an inner edge of the second electrode portion such that the first electrode portion overlaps part of the second electrode portion, the inner edge of the second electrode portion defining the cutout of the second electrode portion.
27. The electrode assembly of any one of clauses 23 to 26, wherein the second electrode portion is disposed on the first electrode portion at a periphery of the first electrode portion, the periphery of the first electrode portion including the edge of the first electrode portion.
28. The electrode assembly of any one of clauses 23 to 27, wherein the second electrode portion is coupled to the first electrode portion with an adhesive.
29. The electrode assembly of any one of clauses 23 to 28, wherein the first electrode portion is removable from the second electrode portion.
30. The electrode assembly of clause 29, wherein the first electrode portion is configured to be used without the second electrode portion to deliver therapy to a patient who is less than a threshold age.
31. The electrode assembly of clause 29 or 30, wherein the first electrode portion is removable from the second electrode portion by peeling the first electrode portion away from the second electrode portion.
32. The electrode assembly of any one of clauses 23 to 31, wherein the first electrode portion comprises a pull tab.
33. The electrode assembly of clause 32, wherein the pull tab is configured to be grasped by a user to peel the first electrode portion away from the second electrode portion.
34. The electrode assembly of any one of clauses 23 to 33, wherein: the first electrode portion comprises a first conductive area; the second electrode portion comprises a second conductive area; the electrode assembly further comprises a layer of gel disposed on the first conductive area and the second conductive area; and the layer of gel is perforated.
35. The electrode assembly of any one of clauses 23 to 34, wherein: the first electrode portion comprises a first conductive area; the second electrode portion comprises a second conductive area; and the first electrode portion and the second electrode portion are configured to be used together to deliver therapy to a patient who is greater than a threshold age.
36. The electrode assembly of clause 35, further comprising: an electrical cable coupled to the first electrode portion at a first end of the electrical cable, wherein the electrical cable is configured to be coupled to a medical device at a second end of the electrical cable; and a layer of gel disposed on the first conductive area and the second conductive area, wherein the layer of gel provides electrical conductivity between the first electrode portion and the second electrode portion.
37. The electrode assembly of clause 35, further comprising: an electrical cable coupled to the first electrode portion at a first end of the electrical cable, wherein the electrical cable is configured to be coupled to a medical device at a second end of the electrical cable; and a conductive element coupling the first conductive area to the second conductive area to provide electrical conductivity between the first electrode portion and the second electrode portion.
38. The electrode assembly of clause 35, further comprising: a first layer of gel disposed on the first conductive area; a conductive film disposed on the first layer of gel; and a second layer of gel disposed on the second conductive area and the conductive film.
39. The electrode assembly of any one of clauses 23 to 38, wherein the cutout is a first cutout, and the edge is a first edge, the electrode assembly further comprising: a third electrode portion disposed on the second electrode portion at a second edge of the second electrode portion, the third electrode portion having a second cutout, and the first electrode portion and the second electrode portion spanning the second cutout.
40. The electrode assembly of clause 39, wherein: the first electrode portion comprises a first conductive area; the second electrode portion comprises a second conductive area; the third electrode portion comprises a third conductive area; and the first electrode portion, the second electrode portion, and the third electrode portion are configured to be used together to deliver therapy to a patient who is greater than a threshold age.
41. The electrode assembly of clause 39, wherein: the first electrode portion comprises a first conductive area and is removable from the second electrode portion and from the third electrode portion and is configured to be used without the second electrode portion and without the third electrode portion to deliver therapy to a first patient who is less than a threshold age; and the second electrode portion comprises a second conductive area and is removable from the third electrode portion and is configured to be used with the first electrode portion and without the third electrode portion to deliver therapy to a second patient who is equal to or greater than the threshold age.
42. A method comprising: removing, based on a size of a patient, a first electrode portion of an electrode assembly from a second electrode portion of the electrode assembly, wherein the second electrode portion has a cutout and was, prior to the removing, disposed on the first electrode portion at an edge of the first electrode portion such that the first electrode portion spanned the cutout; placing the first electrode portion on the patient; and causing delivery of therapy to the patient via a first conductive area of the first electrode portion.
43. The method of clause 42, wherein the removing of the first electrode portion from the second electrode portion based on the size of the patient comprises: measuring a portion of a body of the patient using a measurement scale printed on an electrical cable of the electrode assembly to obtain a measurement; and determining, based on the measurement, that the first electrode portion corresponds to the size of the patient.
44. The method of clause 43, wherein the portion of the body is a demi-span of the body.
45. The method of any one of clauses 42 to 44, wherein the removing of the first electrode portion from the second electrode portion based on the size of the patient comprises: measuring a portion of a body of the patient using a measurement scale printed on an electrical cable of the electrode assembly; determining that the portion of the body is within a portion of the measurement scale that is color-coded with a particular color; and determining that a color of the first electrode portion matches the particular color.
46. The method of clause 45, wherein the portion of the body is a demi-span of the body.
47. The method of any one of clauses 42 to 46, wherein the causing the delivery of the therapy is performed without using the second electrode portion.
48. The method of any one of clauses 42 to 47, wherein the removing comprises peeling the first electrode portion away from the second electrode portion.
49. The method of clause 48, further comprising grasping a pull tab of the first electrode portion prior to the peeling.
50. The method of any one of clauses 42 to 49, wherein the causing the delivery of the therapy comprises causing delivery of an electrical shock to the patient via the first conductive area based on an electrical signal received by the first electrode portion from a medical device.
51. A method comprising: removing, based on a size of a patient, a second electrode portion of an electrode assembly from a third electrode portion of the electrode assembly, wherein the second electrode portion is, after the removing, disposed on a first electrode portion of the electrode assembly at an edge of the first electrode portion, the second electrode portion having a cutout, and the first electrode portion spanning the cutout; placing the first electrode portion and the second electrode portion on the patient; and causing delivery of therapy to the patient via a first conductive area of the first electrode portion and via a second conductive area of the second electrode portion.
52. The method of clause 51, wherein the removing of the second electrode portion from the third electrode portion based on the size of the patient comprises: measuring a portion of a body of the patient using a measurement scale printed on an electrical cable of the electrode assembly to obtain a measurement; and determining, based on the measurement, that the second electrode portion corresponds to the size of the patient.
53. The method of clause 52, wherein the portion of the body is a demi-span of the body.
54. The method of any one of clauses 51 to 53, wherein the removing of the second electrode portion from the third electrode portion based on the size of the patient comprises: measuring a portion of a body of the patient using a measurement scale printed on an electrical cable of the electrode assembly; determining that the portion of the body is within a portion of the measurement scale that is color-coded with a particular color; and determining that a color of the second electrode portion matches the particular color.
55. The method of clause 54, wherein the portion of the body is a demi-span of the body.
56. The method of any one of clauses 51 to 55, wherein the causing the delivery of the therapy is performed without using the third electrode portion.
57. The method of any one of clauses 51 to 56, wherein the removing comprises peeling the second electrode portion away from the third electrode portion.
58. The method of clause 57, further comprising grasping a pull tab of the second electrode portion prior to the peeling.
59. The method of any one of clauses 51 to 58, wherein the causing the delivery of the therapy comprises causing delivery of an electrical shock to the patient via the first conductive area and via the second conductive area based on an electrical signal received by the first electrode portion from a medical device.
60. A method comprising: removing, based on a size of a patient, a third electrode portion of an electrode assembly from a fourth electrode portion of the electrode assembly, wherein the third electrode portion is, after the removing, disposed on a second electrode portion of the electrode assembly at a second edge of the second electrode portion, the third electrode portion having a second cutout, and the second electrode portion spanning the second cutout, and wherein the second electrode portion is, after the removing, disposed on a first electrode portion of the electrode assembly at a first edge of the first electrode portion, the second electrode portion having a first cutout, and the first electrode portion spanning the first cutout; placing the first electrode portion, the second electrode portion, and the third electrode portion on the patient; and causing delivery of therapy to the patient via a first conductive area of the first electrode portion, via a second conductive area of the second electrode portion, and via a third conductive area of the third electrode portion.
61. The method of clause 60, wherein the removing of the third electrode portion from the fourth electrode portion based on the size of the patient comprises: measuring a portion of a body of the patient using a measurement scale printed on an electrical cable of the electrode assembly to obtain a measurement; and determining, based on the measurement, that the third electrode portion corresponds to the size of the patient.
62. The method of clause 61, wherein the portion of the body is a demi-span of the body.
63. The method of any one of clauses 60 to 62, wherein the removing of the third electrode portion from the fourth electrode portion based on the size of the patient comprises: measuring a portion of a body of the patient using a measurement scale printed on an electrical cable of the electrode assembly; determining that the portion of the body is within a portion of the measurement scale that is color-coded with a particular color; and determining that a color of the third electrode portion matches the particular color.
64. The method of clause 63, wherein the portion of the body is a demi-span of the body.
65. The method of any one of clauses 60 to 64, wherein the causing the delivery of the therapy is performed without using the fourth electrode portion.
66. The method of any one of clauses 60 to 65, wherein the removing comprises peeling the third electrode portion away from the fourth electrode portion.
67. The method of clause 66, further comprising grasping a pull tab of the third electrode portion prior to the peeling.
68. The method of any one of clauses 60 to 67, wherein the causing the delivery of the therapy comprises causing the delivery of an electrical shock to the patient via the first conductive area, via the second conductive area, and via the third conductive area based on an electrical signal received by the first electrode portion from a medical device.
69. A method comprising: receiving, by a first electrode portion of an electrode assembly that comprises the first electrode portion and a second electrode portion, an electrical signal from a medical device, wherein the second electrode portion is disposed on the first electrode portion at an edge of the first electrode portion, the second electrode portion has a cutout, and the first electrode portion spans the cutout; and delivering, via a first conductive area of the first electrode portion and via a second conductive area of the second electrode portion, an electrical shock based on the electrical signal received by the first electrode portion from the medical device.
70. The method of clause 69, wherein the delivering of the electrical shock comprises delivering the electrical shock at an energy level associated with a patient who is greater than a threshold age.
71. The method of clause 69 or 70, wherein: the edge is a first edge; the electrode assembly further comprises a third electrode portion disposed on the second electrode portion at a second edge of the second electrode portion, the third electrode portion having a second cutout, and the first electrode portion and the second electrode portion spanning the second cutout; and the delivering of the electrical shock comprises delivering the electrical shock via the first conductive area of the first electrode portion, via the second conductive area of the second electrode portion, and via a third conductive area of the third electrode portion.
72. A method comprising: receiving, by a first electrode portion of an electrode assembly that comprises the first electrode portion and a second electrode portion, an electrical signal from a medical device, wherein the first electrode portion is separated from the second electrode portion, and wherein the second electrode portion was disposed on the first electrode portion at an edge of the first electrode portion prior to separation of the first electrode portion from the second electrode portion, the second electrode portion having a cutout, and wherein the first electrode portion spanned the cutout prior to the separation; and delivering, via a conductive area of the first electrode portion, an electrical shock based on the electrical signal received by the first electrode portion from the medical device.
73. The method of clause 72, wherein the delivering of the electrical shock comprises delivering the electrical shock at an energy level associated with a patient who is less than a threshold age.
74. An external defibrillator comprising: an electrode storage tray; an electrode assembly disposed in the electrode storage tray, the electrode assembly comprising a first electrode portion and second electrode portion; a processor; and memory storing computer-executable instructions that, when executed by the processor, cause performance of operations comprising: determining that the first electrode portion has been removed from the electrode storage tray and that the second electrode portion remains disposed in the electrode storage tray; and selecting, based on the first electrode portion having been removed from the electrode storage tray and the second electrode portion remaining disposed in the electrode storage tray, a pediatric mode in which the external defibrillator is to operate for delivering defibrillation therapy to a patient.
75. The external defibrillator of clause 74, wherein the operations further comprise selecting, based on the pediatric mode, an energy level at which to deliver an electrical shock via the first electrode portion.
76. The external defibrillator of clause 74 or 75, further comprising an output device, wherein the operations further comprise outputting, via the output device, a notification that the external defibrillator is in the pediatric mode.
77. The external defibrillator of any one of clauses 74 to 76, further comprising an input device, and wherein the operations further comprise receiving, via the input device, user input to select an adult mode in which the external defibrillator is to operate for the delivering defibrillation therapy to the patient.
78. The external defibrillator of clause 77, further comprising an output device, wherein the operations further comprise outputting, via the output device: an indication that the adult mode selected by a user of the external defibrillator is incompatible with the first electrode portion that has been removed from the electrode storage tray; and a request for the user to acknowledge that the adult mode is incompatible with the first electrode portion.
79. The external defibrillator of clause 77, further comprising an output device, wherein the operations further comprise outputting, via the output device: an indication that the adult mode selected by a user of the external defibrillator is incompatible with the first electrode portion that has been removed from the electrode storage tray; and a prompt for the user to remove the second electrode portion from the electrode storage tray and to re-attach the second electrode portion to the first electrode portion.
80. The external defibrillator of any one of clauses 74 to 79, further comprising an optical sensor disposed in the electrode storage tray, wherein the determining that the first electrode portion has been removed from the electrode storage tray and that the second electrode portion remains disposed in the electrode storage tray comprises detecting, by the optical sensor, after the first electrode portion has been removed from the electrode storage tray, a visual indicator associated with the second electrode portion.
81. The external defibrillator of clause 80, wherein the electrode assembly further comprises a third electrode portion, wherein the visual indicator is a second visual indicator, and wherein the determining that the first electrode portion has been removed from the electrode storage tray and that the second electrode portion remains disposed in the electrode storage tray comprises: failing to detect, by the optical sensor, a first visual indicator associated with the first electrode portion; and detecting, by the optical sensor, the second visual indicator associated with the second electrode portion; and wherein the operations further comprise determining that the third electrode portion remains disposed in the electrode storage tray by detecting, by the optical sensor, a third visual indicator associated with the third electrode portion.
82. The external defibrillator of any one of clauses 74 to 81, further comprising an electrical circuit disposed in the electrode storage tray, the electrical circuit comprising sense leads, wherein a layer of gel disposed on the second electrode portion forms a conductive bridge between the sense leads to create a closed circuit when the second electrode portion is disposed in the electrode storage tray, and wherein the determining that the first electrode portion has been removed from the electrode storage tray and that the second electrode portion remains disposed in the electrode storage tray comprises detecting the closed circuit after the first electrode portion has been removed from the electrode storage tray.
83. The external defibrillator of clause 82, further comprising: an impedance detection circuit; and a conductive element disposed in the electrode storage tray, and wherein: the electrode assembly further comprises a third electrode portion; the operations further comprise determining that the third electrode portion remains disposed in the electrode storage tray; and the determining that the first electrode portion has been removed from the electrode storage tray and that the second electrode portion and the third electrode portion remain disposed in the electrode storage tray comprises: detecting, by the impedance detection circuit, an impedance value associated with the conductive element; and determining that the impedance value is within a predetermined range of impedance values.
84. The external defibrillator of any one of clauses 74 to 83, further comprising a mechanical switch disposed in the electrode storage tray, the mechanical switch being configured to switch from a first state to a second state in response to removal of the second electrode portion from the electrode storage tray, wherein the determining that the first electrode portion has been removed from the electrode storage tray and that the second electrode portion remains disposed in the electrode storage tray comprises determining, after the first electrode portion has been removed from the electrode storage tray, that the mechanical switch remains in the first state.
85. The external defibrillator of clause 84, wherein the electrode assembly further comprises a third electrode portion, wherein the mechanical switch is a second mechanical switch, the external defibrillator further comprising a first mechanical switch and a third mechanical switch disposed in the electrode storage tray, the first mechanical switch being configured to switch from the first state to the second state in response to removal of the first electrode portion from the electrode storage tray, and the third mechanical switch being configured to switch from the first state to the second state in response to removal of the third electrode portion from the electrode storage tray, and wherein the determining that the first electrode portion has been removed from the electrode storage tray and that the second electrode portion remains disposed in the electrode storage tray comprises determining that: the first mechanical switch has switched to the second state; and the second mechanical switch remains in the first state; and wherein the operations further comprise determining that the third electrode portion remains disposed in the electrode storage tray by determining that the third mechanical switch remains in the first state.
86. A medical device comprising: an electrode storage tray; an electrode assembly disposed in the electrode storage tray, the electrode assembly comprising a first electrode portion and second electrode portion; a processor; and memory storing computer-executable instructions that, when executed by the processor, cause performance of operations comprising: determining that the first electrode portion has been removed from the electrode storage tray and that the second electrode portion remains disposed in the electrode storage tray; and selecting, based on the first electrode portion having been removed from the electrode storage tray and the second electrode portion remaining disposed in the electrode storage tray, a usage mode of a plurality of usage modes in which the medical device is to operate.
87. The medical device of clause 86, wherein the operations further comprise selecting, based on the usage mode, an energy level associated with an electrical signal to be sent to the first electrode portion.
88. The medical device of clause 86 or 87, further comprising an output device, wherein the operations further comprise outputting, via the output device, a notification that the medical device is in the usage mode.
89. The medical device of any one of clauses 86 to 88, further comprising an input device, wherein the usage mode is a first usage mode, and wherein the operations further comprise receiving, via the input device, user input to select a second usage mode of the plurality of usage modes in which the medical device is to operate.
90. The medical device of clause 89, further comprising an output device, wherein the operations further comprise outputting, via the output device: an indication that the second usage mode selected by a user of the medical device is incompatible with the first electrode portion that has been removed from the electrode storage tray; and a request for the user to acknowledge that the second usage mode is incompatible with the first electrode portion.
91. The medical device of clause 89, further comprising an output device, wherein the operations further comprise outputting, via the output device: an indication that the second usage mode selected by a user of the medical device is incompatible with the first electrode portion that has been removed from the electrode storage tray; and a prompt for the user to remove the second electrode portion from the electrode storage tray and to re-attach the second electrode portion to the first electrode portion.
92. The medical device of any one of clauses 86 to 91, further comprising an optical sensor disposed in the electrode storage tray, wherein the determining that the first electrode portion has been removed from the electrode storage tray and that the second electrode portion remains disposed in the electrode storage tray comprises detecting, by the optical sensor, after the first electrode portion has been removed from the electrode storage tray, a visual indicator associated with the second electrode portion.
93. The medical device of clause 92, wherein the electrode assembly further comprises a third electrode portion, wherein the visual indicator is a second visual indicator, and wherein the determining that the first electrode portion has been removed from the electrode storage tray and that the second electrode portion remains disposed in the electrode storage tray comprises: failing to detect, by the optical sensor, a first visual indicator associated with the first electrode portion; and detecting, by the optical sensor, the second visual indicator associated with the second electrode portion; and wherein the operations further comprise determining that the third electrode portion remains disposed in the electrode storage tray by detecting, by the optical sensor, a third visual indicator associated with the third electrode portion.
94. The medical device of any one of clauses 86 to 93, further comprising an electrical circuit disposed in the electrode storage tray, the electrical circuit comprising sense leads, wherein a layer of gel disposed on the second electrode portion forms a conductive bridge between the sense leads to create a closed circuit when the second electrode portion is disposed in the electrode storage tray, and wherein the determining that the first electrode portion has been removed from the electrode storage tray and that the second electrode portion remains disposed in the electrode storage tray comprises detecting the closed circuit after the first electrode portion has been removed from the electrode storage tray.
95. The medical device of clause 94, further comprising: an impedance detection circuit; and a conductive element disposed in the electrode storage tray, and wherein: the electrode assembly further comprises a third electrode portion; the operations further comprise determining that the third electrode portion remains disposed in the electrode storage tray; and the determining that the first electrode portion has been removed from the electrode storage tray and that the second electrode portion and the third electrode portion remain disposed in the electrode storage tray comprises: detecting, by the impedance detection circuit, an impedance value associated with the conductive element; and determining that the impedance value is within a predetermined range of impedance values.
96. The medical device of any one of clauses 86 to 95, further comprising a mechanical switch disposed in the electrode storage tray, the mechanical switch being configured to switch from a first state to a second state in response to removal of the second electrode portion from the electrode storage tray, wherein the determining that the first electrode portion has been removed from the electrode storage tray and that the second electrode portion remains disposed in the electrode storage tray comprises determining, after the first electrode portion has been removed from the electrode storage tray, that the mechanical switch remains in the first state.
97. The medical device of clause 96, wherein the electrode assembly further comprises a third electrode portion, wherein the mechanical switch is a second mechanical switch, the medical device further comprising a first mechanical switch and a third mechanical switch disposed in the electrode storage tray, the first mechanical switch being configured to switch from the first state to the second state in response to removal of the first electrode portion from the electrode storage tray, and the third mechanical switch being configured to switch from the first state to the second state in response to removal of the third electrode portion from the electrode storage tray, and wherein the determining that the first electrode portion has been removed from the electrode storage tray and that the second electrode portion remains disposed in the electrode storage tray comprises determining that: the first mechanical switch has switched to the second state; and the second mechanical switch remains in the first state; and wherein the operations further comprise determining that the third electrode portion remains disposed in the electrode storage tray by determining that the third mechanical switch remains in the first state.
98. A method comprising: determining, by a medical device, that a first electrode portion of an electrode assembly has been removed from an electrode storage tray of the medical device and that a second electrode portion of the electrode assembly remains disposed in the electrode storage tray; and selecting, by the medical device, and based on the first electrode portion having been removed from the electrode storage tray and the second electrode portion remaining disposed in the electrode storage tray, a usage mode of a plurality of usage modes in which the medical device is to operate.
99. The method of clause 98, further comprising selecting, by the medical device, and based on the usage mode, an energy level associated with an electrical signal to be sent to the first electrode portion.
100. The method of clause 98 or 99, further comprising outputting, via an output device of the medical device, a notification that the medical device is in the usage mode.
101. The method of any one of clauses 98 to 100, wherein the usage mode is a first usage mode, the method further comprising receiving, via an input device of the medical device, user input to select a second usage mode of the plurality of usage modes in which the medical device is to operate.
102. The method of clause 101, further comprising outputting, via an output device of the medical device: an indication that the second usage mode selected by a user of the medical device is incompatible with the first electrode portion that has been removed from the electrode storage tray; and a request for the user to acknowledge that the second usage mode is incompatible with the first electrode portion.
103. The method of clause 101, further comprising outputting, via an output device of the medical device: an indication that the second usage mode selected by a user of the medical device is incompatible with the first electrode portion that has been removed from the electrode storage tray; and a prompt for the user to remove the second electrode portion from the electrode storage tray and to re-attach the second electrode portion to the first electrode portion.
104. The method of any one of clauses 98 to 103, wherein the determining that the first electrode portion has been removed from the electrode storage tray and that the second electrode portion remains disposed in the electrode storage tray comprises detecting, by an optical sensor disposed within the electrode storage tray, after the first electrode portion has been removed from the electrode storage tray, a visual indicator associated with the second electrode portion.
105. The method of clause 104, wherein the visual indicator is a second visual indicator, and wherein the determining that the first electrode portion has been removed from the electrode storage tray and that the second electrode portion remains disposed in the electrode storage tray comprises: failing to detect, by the optical sensor, a first visual indicator associated with the first electrode portion; and detecting, by the optical sensor, the second visual indicator associated with the second electrode portion; and the method further comprising determining, by the medical device, that a third electrode portion of the electrode assembly remains disposed in the electrode storage tray by detecting, by the optical sensor, a third visual indicator associated with the third electrode portion.
106. The method of any one of clauses 98 to 105, wherein the determining that the first electrode portion has been removed from the electrode storage tray and that the second electrode portion remains disposed in the electrode storage tray comprises detecting, after the first electrode portion has been removed from the electrode storage tray, a closed circuit created by a layer of gel disposed on the second electrode portion forming a conductive bridge between sense leads of an electrical circuit disposed in the electrode storage tray.
107. The method of clause 106, further comprising: determining, by the medical device, that a third electrode portion of the electrode assembly remains disposed in the electrode storage tray, and wherein the determining that the first electrode portion has been removed from the electrode storage tray and that the second electrode portion and the third electrode portion remain disposed in the electrode storage tray comprises: detecting, by an impedance detection circuit of the medical device, an impedance value associated with a conductive element disposed in the electrode storage tray; and determining that the impedance value is within a predetermined range of impedance values.
108. The method of any one of clauses 98 to 107, wherein the determining that the first electrode portion has been removed from the electrode storage tray and that the second electrode portion remains disposed in the electrode storage tray comprises determining, after the first electrode portion has been removed from the electrode storage tray, that a mechanical switch disposed in the electrode storage tray and associated with the second electrode portion remains in a first state of two states.
109. The method of clause 108, wherein the mechanical switch is a second mechanical switch, and wherein the determining that the first electrode portion has been removed from the electrode storage tray and that the second electrode portion remains disposed in the electrode storage tray comprises determining that: a first mechanical switch disposed in the electrode storage tray and associated with the first electrode portion has switched to a second state of the two states; and the second mechanical switch remains in the first state; and the method further comprising determining that a third electrode portion of the electrode assembly remains disposed in the electrode storage tray by determining that a third mechanical switch disposed in the electrode storage tray and associated with the third electrode portion remains in the first state.
110. An external defibrillator comprising: an electrode storage tray; an electrode assembly disposed in the electrode storage tray, the electrode assembly comprising a first electrode portion and second electrode portion; a processor; and memory storing computer-executable instructions that, when executed by the processor, cause performance of operations comprising: determining that the first electrode portion and the second electrode portion have been removed from the electrode storage tray; and selecting, based on the first electrode portion having been removed from the electrode storage tray and the second electrode portion remaining disposed in the electrode storage tray, an adult mode of a plurality of usage modes in which the external defibrillator is to operate for delivering defibrillation therapy to a patient.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing the disclosed techniques and systems in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

1. An electrode assembly comprising:
a first electrode portion; and
a second electrode portion disposed on the first electrode portion at an edge of the first electrode portion, the second electrode portion having a cutout, and the first electrode portion spanning the cutout.

2. The electrode assembly of claim 1, wherein the cutout is centered within the second electrode portion.

3. The electrode assembly of claim 1, wherein the second electrode portion extends beyond the edge of the first electrode portion.

4. The electrode assembly of claim 1, wherein the edge of the first electrode portion is an outer edge of the first electrode portion, and wherein the outer edge of the first electrode portion is horizontally offset from an inner edge of the second electrode portion such that the first electrode portion overlaps part of the second electrode portion, the inner edge of the second electrode portion defining the cutout of the second electrode portion.

5. The electrode assembly of claim 1, wherein the second electrode portion is disposed on the first electrode portion at a periphery of the first electrode portion, the periphery of the first electrode portion including the edge of the first electrode portion.

6. The electrode assembly of claim 1, wherein the second electrode portion is coupled to the first electrode portion with an adhesive.

7. The electrode assembly of claim 1, wherein the first electrode portion is removable from the second electrode portion.

8. The electrode assembly of claim 7, wherein the first electrode portion is configured to be used without the second electrode portion to deliver therapy to a patient who is less than a threshold age.

9. The electrode assembly of claim 7, wherein the first electrode portion is removable from the second electrode portion by peeling the first electrode portion away from the second electrode portion.

10. The electrode assembly of claim 1, wherein the first electrode portion comprises a pull tab configured to be grasped by a user to peel the first electrode portion away from the second electrode portion.

11. The electrode assembly of claim 1, wherein:
the first electrode portion comprises a first conductive area;
the second electrode portion comprises a second conductive area;
the electrode assembly further comprises a layer of gel disposed on the first conductive area and the second conductive area; and
the layer of gel is perforated.

12. The electrode assembly of claim 1, wherein:
the first electrode portion comprises a first conductive area;
the second electrode portion comprises a second conductive area; and
the first electrode portion and the second electrode portion are configured to be used together to deliver therapy to a patient who is greater than a threshold age.

13. The electrode assembly of claim 12, further comprising:
an electrical cable coupled to the first electrode portion at a first end of the electrical cable, wherein the electrical cable is configured to be coupled to a medical device at a second end of the electrical cable; and
a layer of gel disposed on the first conductive area and the second conductive area,
wherein the layer of gel provides electrical conductivity between the first electrode portion and the second electrode portion.

14. The electrode assembly of claim 12, further comprising:
an electrical cable coupled to the first electrode portion at a first end of the electrical cable, wherein the electrical cable is configured to be coupled to a medical device at a second end of the electrical cable; and
a conductive element coupling the first conductive area to the second conductive area to provide electrical conductivity between the first electrode portion and the second electrode portion.

15. The electrode assembly of claim 12, further comprising:
a first layer of gel disposed on the first conductive area;
a conductive film disposed on the first layer of gel; and
a second layer of gel disposed on the second conductive area and the conductive film.

16. A method comprising:
receiving, by a first electrode portion of an electrode assembly that comprises the first electrode portion and a second electrode portion, an electrical signal from a medical device, wherein the second electrode portion is disposed on the first electrode portion at an edge of the first electrode portion, the second electrode portion has a cutout, and the first electrode portion spans the cutout; and
delivering, via a first conductive area of the first electrode portion and via a second conductive area of the second electrode portion, an electrical shock based on the electrical signal received by the first electrode portion from the medical device.

17. The method of claim 16, wherein the delivering of the electrical shock comprises delivering the electrical shock at an energy level associated with a patient who is greater than a threshold age.

18. The method of claim 16, wherein:
the edge is a first edge;
the electrode assembly further comprises a third electrode portion disposed on the second electrode portion at a second edge of the second electrode portion, the third electrode portion having a second cutout, and the first electrode portion and the second electrode portion spanning the second cutout; and
the delivering of the electrical shock comprises delivering the electrical shock via the first conductive area of the first electrode portion, via the second conductive area of the second electrode portion, and via a third conductive area of the third electrode portion.

19. A method comprising:
receiving, by a first electrode portion of an electrode assembly that comprises the first electrode portion and a second electrode portion, an electrical signal from a medical device, wherein the first electrode portion is separated from the second electrode portion, and wherein the second electrode portion was disposed on the first electrode portion at an edge of the first electrode portion prior to separation of the first electrode portion from the second electrode portion, the second electrode portion having a cutout, and wherein the first electrode portion spanned the cutout prior to the separation; and
delivering, via a conductive area of the first electrode portion, an electrical shock based on the electrical signal received by the first electrode portion from the medical device.

20. The method of claim 20, wherein the delivering of the electrical shock comprises delivering the electrical shock at an energy level associated with a patient who is less than a threshold age.
